(19) United States
(12) Patent Application Publication
(10) Pub. No.: US 2004/0220631 A1
(43) Pub. Date: Nov. 4, 2004

(54) Method and apparatus for detecting myocardial electrical recovery and controlling extra-systolic sstimulation

(75) Inventors: John E. Burnes, Andover, MN (US); Nirav Vijay Sheth, Coon Rapids, MN (US); Chris Zillmer, Hudson, WI (US); Vincent E. splett, Apple Valley, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/426,613

(22) Filed: Apr. 29, 2003

(51) Int. Cl. A61N 1/362

(52) U.S. Cl. 607/9

(57) ABSTRACT

An implantable system and method are provided for detecting myocardial electrical recovery time and for controlling the delivery of cardiac stimulation pulses relative to the detected recovery time. One or more fiducial points on a T-wave signal sensed from one or more intracardiac EGM or subcutaneous ECG signals are detected for estimating myocardial recovery time. Extra systolic stimulation pulses are delivered at an extra systolic interval based on a detected recovery time to effectively produce post-extra systolic potentiation while preventing delivery of extra systolic stimuli during the vulnerable period.

### CROSS REFERENCE TO RELATED APPLICATIONS

This patent disclosure hereby incorporates by reference the following patent applications filed on even date hereof; namely, P-11030, “Cardiac Pacing Therapy Parameter Programming”; P-11216, “Method and Apparatus to Monitor Pulmonary Edema; P-11252, “Method and Apparatus for Determining Myocardial Electrical Resitution and Controlling Extra Systolic Stimulation; and P-11215, “Use of Activation and Recovery Times and Dispersions to Monitor Heart Failure Status and Arrhythmia Risk”.

### FIELD OF THE INVENTION

The present invention relates generally to the field of implantable cardiac stimulation devices and more specifically to a device and method for detecting myocardial recovery time and for delivering extra systolic stimulation relative to a detected recovery time to safely achieve post-extra systolic potentiation.

### BACKGROUND OF THE INVENTION

Post-extra systolic potentiation (PESP) is a property of cardiac myocytes that results in enhanced mechanical function of the heart on the beats following an extra systolic stimulus delivered early after either an intrinsic or pacing-induced systole. The magnitude of the enhanced mechanical function is strongly dependent on the timing of the extra systole relative to the preceding intrinsic or paced systole. When correctly timed, an extra systolic stimulation pulse causes an electrical depolarization of the heart but the attendant mechanical contraction is absent or substantially weakened. The contractility of the subsequent cardiac cycles, referred to as the post-extra systolic beats, is increased as described in detail in commonly assigned U.S. Pat. No. 5,213,098 issued to Bennett et al., incorporated herein by reference in its entirety.

The mechanism of PESP is thought to involve the calcium cycling within the myocytes. The extra systole initiates a limited calcium release from the sarcolasmic reticulum (SR). The limited amount of calcium that is released in response to the extra systole is not enough to cause a normal mechanical contraction of the heart. After the extra systole, the SR continues to take up calcium with the result that subsequent depolarization(s) cause a large release of calcium from the SR, resulting in vigorous myocyte contraction.

As noted, the degree of mechanical augmentation on post-extra systolic beats depends strongly on the time interval between a primary systole and the subsequent extra systole, referred to herein as the “extra systolic interval” (ESI). If the ESI is too long, the PESP effects are not achieved because a normal mechanical contraction takes place in response to the extra systolic stimulus. As the ESI is shortened, a maximal effect is reached when the ESI is slightly longer than the myocardial refractory period. An electrical depolarization occurs without a mechanical contraction or with a substantially weakened contraction. When the ESI becomes too short, the stimulus falls with the absolute refractory period and no depolarization occurs.

As indicated in the referenced '098 patent, one risk associated with delivering extra systolic stimulation pulses to achieve PESP is arrhythmia induction. If the extra systolic pulse is delivered to cardiac cells during the vulnerable period, the risk of inducing tachycardia or fibrillation in arrhythmia-prone patients is high. The vulnerable period encompasses the repolarization phase of the action potential, also referred to herein as the “recovery phase”, and a period immediately following it. During the vulnerable period, the cardiac cell membrane is transiently hyper-excitable. Therefore, although the property of PESP has been known of for decades, the application of PESP in a cardiac stimulation therapy for improving the mechanical function of the heart has not been realized clinically because of the perceived risks.

The T-wave contains the repolarization or recovery information in a cardiac electrogram (EGM) signal. An EGM signal represents the summation of the action potentials from a myocardial mass, the size of which depends on the sensing electrode configuration. Determination of a local activation-recovery interval (ARI) from a unipolar EGM signal is closely correlated to the duration of the local monophasic action potential. A bipolar sensor for muscle tissue action potential duration estimation is generally disclosed in U.S. Pat. No. 6,522,904 issued to Mika et al. With the development of modern sense amplifiers and digital signal analysis, reliable T-wave sensing is feasible. T-wave sensing for indicating the end of the cardiac refractory period has been proposed for use in other types of cardiac therapies, for example in properly timing the delivery of anti-tachycardia pacing therapies. Reference is made to U.S. Pat. No. 4,593,695 issued to Wittkampf. In U.S. Pat. No. 5,899,929 issued to Thompson et al., and in U.S. Pat. No. 6,052,621 issued to Begemann et al., systems for tachycardia induction employing T-wave sensing are generally disclosed. Stimulation pulses delivered during the T-wave for intentionally inducing arrhythmias are applied during electrophysiological testing performed to determine the optimal anti-tachycardia stimulation therapies for a particular patient.

The above-disclosed systems generally sense a T-wave following an evoked depolarization. However, it is desirable to sense a T-wave following both evoked and intrinsic depolarizations, and more specifically estimate myocardial recovery time from a sensed T-wave. Measurement of the myocardial recovery time may be useful in a number of therapy delivery and diagnostic applications including those mentioned above (extra systolic stimulation for achieving PESP, timing of anti-tachycardia pacing pulses, timing of arrhythmia induction pulses) and others. Changes in the repolarization properties reflected in the morphology or timing of the T-wave or the S-T segment of an EGM or ECG signal may reflect changes in the disease state or physiological condition of the patient. For example, T-wave and S-T segment changes are known to occur with myocardial ischemia or myocardial infarction. A method for determining variation of S-T segment parameters using multiple cardiac electrogram signal vectors for determining physiological conditions such as ischemia is generally disclosed in U.S. Pat. No. 6,128,526 issued to Stadler et al., and in U.S. Pat. No. 6,397,100 issued to Stadler et al.

A different approach to modulating cardiac contractility involves application of non-excitatory electrical stimulation (NES) by excitable tissue controllers (ETCs). A detailed description of ETCs and NES is provided in U.S. Pat. No. 6,360,126 issued to Mika et al. NES is delivered during refractory and is thought to cause catecholamine release within the heart, which in turn increases the contractility of the cardiac tissue. Successful application of NES is also dependent on accurate timing of the NES pulse, but in this approach stimuli are delivered during the local action potential, prior to the end of refractory. Stimuli delivered just after the absolute refractory period, during the vulnerable period, may be arrhythmogenic. A method for automatically controlling the delivery of excitable tissue control signals that includes determination of an estimated action potential duration is generally disclosed in the above referenced '126 patent.

The effects of PESP may advantageously benefit a large number of patients suffering from cardiac mechanical insufficiency, such as patients in heart failure. A need remains therefore for a clinically safe method for delivering an extra systolic stimulation (ESS) therapy that achieves the mechanical benefits of PESP and avoids the risk of arrhythmia induction. Such a method preferably allows ESS pulses to be delivered relative to a known recovery time. Furthermore, determination of myocardial recovery time is useful in controlling the timing of cardiac stimulation pulses in other stimulation applications and in monitoring for changes in electrical recovery that may be indicative of a change in clinical status.

### SUMMARY OF THE INVENTION

The present invention provides a system and method for detecting myocardial recovery time and for delivering extra systolic stimuli at extra systolic intervals for achieving the benefits of PESP while minimizing or eliminating the risk of arrhythmia induction. The system includes an implantable cardiac stimulation device and associated leads for sensing cardiac signals and delivering electrical stimulation pulses to the myocardium. The device includes input, output and control circuitry for receiving cardiac signals, processing received signals for detecting myocardial recovery time, and delivering extra systolic stimulation pulses relative to the detected recovery time.

In a method for detecting myocardial recovery time, one or more fiducial points on one or more T-waves sensed from intracardiac EGM and/or subcutaneous ECG signals are identified. The time corresponding to the one or more fiducial points is used in estimating myocardial recovery time. Fiducial points may be detected based on threshold crossings of sensed T-wave signals and/or morphological features of the T-waves identified through digital signal processing methods. Preferably, fiducial points are detected from a segment of the EGM/ECG signal that includes the T-wave, defined as a “recovery time detection window”. A calibration procedure may be used for setting the recovery time detection window, selecting EGM/ECG sensing vectors to be used for detecting recovery time, and/or selecting T-wave fiducial points used for detecting recovery time.

In one embodiment, multiple fiducial points are detected and the corresponding times are corroborated to estimate a recovery time. If large discrepancies between magnitudes or times of occurrence of fiducial points exist from one cardiac cycle to the next, the recovery time is not detected for the given cardiac cycle. Major changes in T-wave morphology identified by shifts in the magnitude and/or time of fiducial points may be stored for diagnostic purposes or used by the implanted device in controlling a therapy delivery.

A method for controlling the delivery of extra systolic stimulation includes detecting a myocardial recovery time and setting an extra systolic interval (ESI) based on the detected recovery time. In one embodiment, the ESI is set as a fixed interval based on a measured activation-recovery interval (ARI) plus a predetermined safety interval to ensure the extra systolic stimuli occur after the vulnerable period. The ARI is measured as the difference between myocardial activation time and detected recovery time. Activation time may be measured as the time of a fiducial point on a primary intrinsic or pacing-evoked R-wave. Alternatively, the ESI following a pacing-evoked systole may be set based on the time interval measured between a pacing pulse and the detected recovery time plus a predetermined safety interval. In another embodiment, a dynamic ESI is applied by performing beat-by-beat detection of the recovery time and delivering extra systolic stimuli at a predetermined safety interval after the detected recovery time for each beat.

In yet another embodiment, a “look-up” table of ESIs is generated by measuring the ARI for a number of different heart rates and storing the ESI for a given heart rate as the sum of the corresponding ARI and a safety interval. During ESS, the ESI is automatically adjusted to the stored ESI in the “look-up” table in response to changes in heart rate.

The methods of the present invention may further include sensing one or more physiological signals representative of mechanical heart function. The ESI may be optimized based on maximizing the mechanical heart function on post-extra systolic beats. A minimum allowable ESI is limited based on recovery time determination.

In another embodiment, extra systolic stimuli are delivered at multiple sites. The ESIs are automatically adjusted to maintain stimuli delivery safely after the recovery time at each site. Additionally, multi-site cardiac pacing intervals and/or ESIs are automatically adjusted to maintain or reduce the dispersion of recovery time between the multiple sites during ESS.

In yet another embodiment, atrial ESS is delivered to achieve PESP in the atria and ventricles. If the atrial systole and atrial extra systole are both conducted to the ventricles, and if the conducted extra systole occurs during a ventricular ARI plus safety interval, then the atrial ESI may be lengthened or atrial ESS could be disabled to prevent a conducted extra systolic depolarization to occur during the ventricular vulnerable period.

The present invention advantageously allows myocardial recovery time to be detected in an implanted cardiac stimulation/monitoring device and changes in myocardial recovery to be tracked. Aspects of the present invention further allow extra systolic stimulation to be safely delivered such that the benefits of enhanced mechanical function due to PESP may be realized in clinical treatments for cardiac mechanical insufficiency with little or no risk of arrhythmia induction. Furthermore, aspects of the present invention used for detecting myocardial recovery time may be employed in controlling the delivery of stimulation pulses in other cardiac stimulation therapies or diagnostic procedures such as NES, anti-tachycardia pacing or tachycardia induction.

### BRIEF DESCRIPTION OF THE DRAWINGS

FIG. 1A is an illustration of an exemplary cardiac stimulation device, referred to herein as an “implantable medical device” or “IMD,” in which the present invention may be implemented.

FIG. 1B is an illustration of an alternative IMD coupled to a set of leads implanted in a patient's heart.

FIG. 2A is a functional schematic diagram of the implantable medical device shown in FIG. 1A.

FIG. 2B is a functional schematic diagram of an alternative embodiment of the IMD of FIG. 1B, which includes dedicated circuitry for detecting myocardial recovery time.

FIG. 3A is a high-level block diagram of the recovery time detection circuitry included in the IMD of FIG. 2B.

FIG. 3B is a functional block diagram summarizing signal processing operations performed by the T-wave feature detector of FIG. 3A.

FIG. 4A is an illustration of a representative cardiac action potential signal and a unipolar EGM signal, which may be used for detecting myocardial recovery time.

FIG. 4B is a sample graph of measured activation recovery intervals (ARIs) based on detecting recovery time according to three different fiducial points on sensed T-waves at varying heart rates.

FIG. 5 is a flow chart summarizing steps included in a calibration method for validating an ARI measurement.

FIG. 6A is an alternative calibration method that may be used for validating an ARI measurement and/or setting a recovery time sensing window and/or safety interval.

FIG. 6B is a flow chart summarizing steps included in one method for controlling the delivery of ESS based on measuring end refractory time.

FIG. 7 is a flow diagram providing a general overview of operations included in the present invention for controlling the delivery of extra systolic stimulation relative to a measured recovery time.

FIG. 8 is a flow chart summarizing an alternative method for dynamically controlling the delivery of extra systolic stimuli based on the detection of recovery time on a beat-by-beat basis.

FIG. 9A is a timing diagram shown in temporal relation to an EGM signal to illustrate methods for detecting activation and recovery times and controlling ESS.

FIG. 9B is an illustration of a representative EGM signal and a timing diagram illustrating the operation of ESS during paired pacing.

FIG. 10 is a flow diagram of yet another embodiment for controlling the delivery of ESS based on detection of recovery time using a far-field EGM or subcutaneous ECG signal.

FIG. 11 is a flow chart summarizing steps included in a method for automatically adjusting an ESI based on both recovery time and one or more indices of mechanical heart function, which may be hemodynamic indices or indices of myocardial contraction performance.

FIG. 12 is a flow chart summarizing a method for applying multi-site ESS in which relative recovery times between stimulation sites are maintained or recovery time dispersion is reduced.

FIG. 13 is a flow chart summarizing a method for controlling ESS according to previously determined ESIs based on ARIs measured over a range of heart rates.

FIG. 14 is a flow chart summarizing steps included in a method for controlling the delivery of atrial ESS.

FIG. 15 is a flow chart summarizing steps included in a method for controlling the delivery of non-excitatory stimulation pulses based on detecting recovery time.

### DETAILED DESCRIPTION OF THE INVENTION

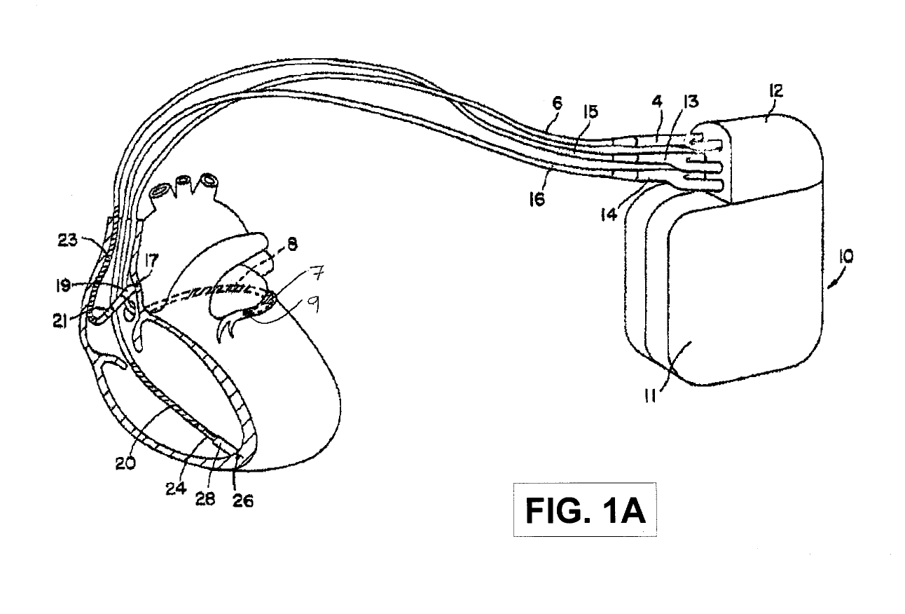

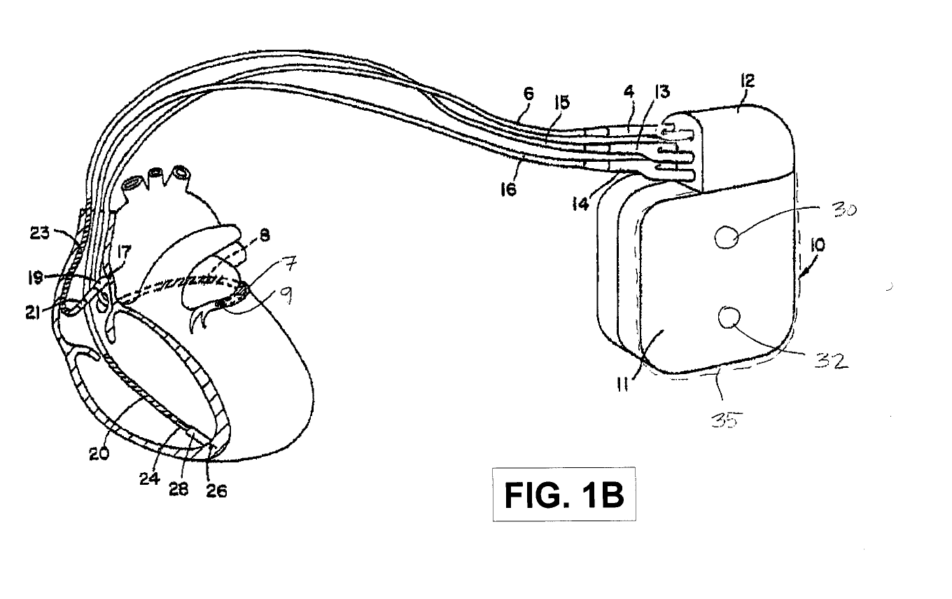

The present invention is directed toward providing an implantable system for detecting a myocardial recovery time and for delivering extra systolic stimulation pulses at a time interval relative to the recovery time. FIG. 1A is an illustration of an exemplary cardiac stimulation device, referred to herein as an “implantable medical device” or “IMD,” in which the present invention may be implemented. IMD 10 is coupled to a patient's heart by three cardiac leads. IMD 10 is capable of receiving cardiac signals and delivering electrical pulses for cardiac pacing, cardioversion and defibrillation. IMD 10 includes a connector block 12 for receiving the proximal end of a right ventricular lead 16, a right atrial lead 15 and a coronary sinus lead 6, used for positioning electrodes for sensing and stimulating in three or four heart chambers.

In FIG. 1A, the right ventricular lead 16 is positioned such that its distal end is in the right ventricle for sensing right ventricular cardiac signals and delivering electrical stimulation therapies in the right ventricle which includes at least ESS and may include bradycardia pacing, cardiac resynchronization therapy, cardioversion and/or defibrillation. For these purposes, right ventricular lead 16 is equipped with a ring electrode 24, a tip electrode 26, optionally mounted retractably within an electrode head 28, and a coil electrode 20, each of which are connected to an insulated conductor within the body of lead 16. The proximal end of the insulated conductors are coupled to corresponding connectors carried by bifurcated connector 14 at the proximal end of lead 16 for providing electrical connection to IMD 10.

The right atrial lead 15 is positioned such that its distal end is in the vicinity of the right atrium and the superior vena cava. Lead 15 is equipped with a ring electrode 21, a tip electrode 17, optionally mounted retractably within electrode head 19, and a coil electrode 23 for providing sensing and electrical stimulation therapies in the right atrium, which may include ESS and other cardiac stimulation therapies, such as bradycardia pacing, cardiac resynchronization therapy, anti-tachycardia pacing, high-voltage cardioversion and/or defibrillation. In one application of PESP, ESS is delivered in the atrial chambers to improve the atrial contribution to ventricular filling. The extra systolic depolarization resulting from the atrial ESS stimulation pulse may be conducted to the ventricles for achieving PESP effects in both the atrial and ventricular chambers. The ring electrode 21, the tip electrode 17 and the coil electrode 23 are each connected to an insulated conductor with the body of the right atrial lead 15. Each insulated conductor is coupled at its proximal end to a connector carried by bifurcated connector 13.

The coronary sinus lead 6 is advanced within the vasculature of the left side of the heart via the coronary sinus and great cardiac vein. The coronary sinus lead 6 is shown in the embodiment of FIG. 1A as having a defibrillation coil electrode 8 that may be used in combination with either the coil electrode 20 or the coil electrode 23 for delivering electrical shocks for cardioversion and defibrillation therapies. Coronary sinus lead 6 is also equipped with a distal tip electrode 9 and ring electrode 7 for pacing and sensing functions and delivering ESS in the left ventricle of the heart. The coil electrode 8, tip electrode 9 and ring electrode 7 are each coupled to insulated conductors within the body of lead 6, which provides connection to the proximal bifurcated connector 4. In alternative embodiments, lead 6 may additionally include ring electrodes positioned for left atrial sensing and stimulation functions, which may include ESS and/or other cardiac stimulation therapies.

The electrodes 17 and 21, 24 and 26, and 7 and 9 may be used in sensing and stimulation as bipolar pairs, commonly referred to as a “tip-to-ring” configuration, or individually in a unipolar configuration with the device housing 11 serving as the indifferent electrode, commonly referred to as the “can” or “case” electrode. Preferably, IMD 10 is capable of delivering high-voltage cardioversion and defibrillation therapies. As such, device housing 11 may also serve as a subcutaneous defibrillation electrode in combination with one or more of the defibrillation coil electrodes 8, 20 or 23 for defibrillation of the atria or ventricles.

For the purposes of detecting myocardial recovery time in accordance with the present invention, an EGM signal may be sensed from a bipolar “tip-to-ring” sensing vector, a unipolar tip-to-can sensing vector, a unipolar tip-to-coil or ring-to-coil sensing vector, or a relatively more global coil-to-can sensing vector. Any combination of available electrodes may be selected for sensing an EGM signal for detecting recovery time. As will be described in greater detail below, a fiducial point on the sensed EGM signal is used to identify a recovery time, preferably correlated to the recovery time at the site at which ESS will be delivered,

It is recognized that alternate lead systems may be substituted for the three lead system illustrated in FIG. 1A. For example, lead systems including one or more unipolar, bipolar and/or mulitpolar leads may be configured for sensing an EGM signal from which a recovery time may be derived and for delivering ESS. Furthermore, epicardial leads could be substituted for transvenous leads. It is contemplated that extra systolic stimuli may be delivered at one or more sites within the heart. Accordingly, lead systems may be adapted for sensing an EGM signal for detecting recovery times at multiple cardiac sites and for delivering extra systolic stimuli at the multiple sites. It is further contemplated that subcutanteous ECG electrodes could be included in the implantable system and that recovery times may be estimated from the subcutaneous ECG signals.

FIG. 1B is an illustration of an alternative IMD coupled to a set of leads implanted in a patient's heart. In FIG. 1B, IMD housing 11 is provided with an insulative coating 35, covering at least a portion of housing 11, with openings 30 and 32. The uninsulated openings 30 and 32 serve as subcutaneous electrodes for sensing global ECG signals, which may be used, in accordance with the present invention, for detecting myocardial recovery. An implantable system having electrodes for subcutanteous measurement of an ECG is generally disclosed in commonly assigned U.S. Pat. No. 5,987,352 issued to Klein, incorporated herein by reference in its entirety. In alternative embodiments, multiple subcutaneous electrodes incorporated on the device housing 11 and/or positioned on subcutaneous leads extending from IMD 10 may be used to acquire multiple subcutaneous ECG sensing vectors for measurement myocardial recovery. Multi-electrode ECG sensing in an implantable monitor is described in U.S. Pat. No. 5,313,953 issued to Yomtov, et al., incorporated herein by reference in its entirety.

While a particular multi-chamber IMD and lead system is illustrated in FIGS. 1A and 1B, methodologies included in the present invention may be adapted for use with other single chamber, dual chamber, or multichamber cardiac stimulation devices that are intended for delivering ESS and may optionally include other electrical stimulation therapy delivery capabilities such as bradycardia pacing, cardiac resynchronization therapy, anti-tachycardia pacing, high-voltage cardioversion, and/or defibrillation.

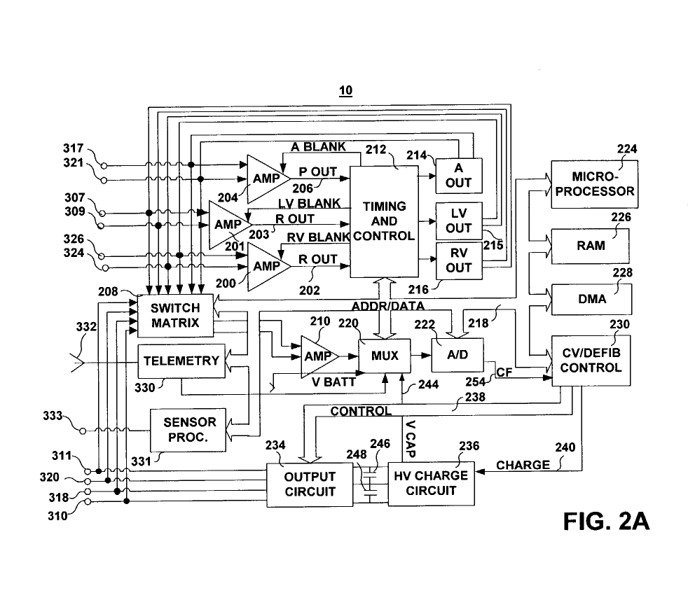

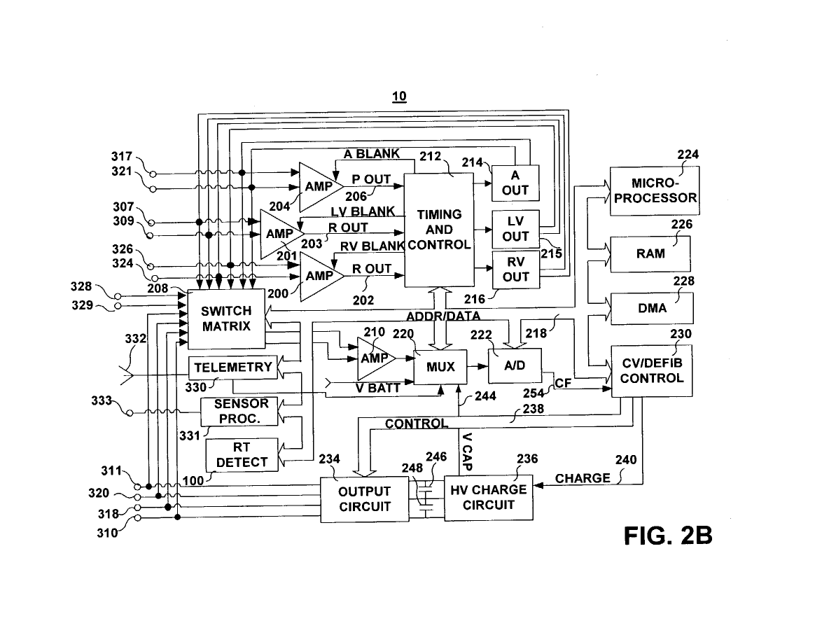

A functional schematic diagram of IMD 10 is shown in FIG. 2A. This diagram should be taken as exemplary of the type of device in which the invention may be embodied and not as limiting. The disclosed embodiment shown in FIG. 2A is a microprocessor-controlled device, but the methods of the present invention may also be practiced in other types of devices such as those employing dedicated digital circuitry.

With regard to the electrode system illustrated in FIG. 1A, IMD 10 is provided with a number of connection terminals for achieving electrical connection to the leads 6, 15, and 16 and their respective electrodes. The connection terminal 311 provides electrical connection to the housing 11 for use as the indifferent electrode during unipolar stimulation or sensing. The connection terminals 320, 310, and 318 provide electrical connection to coil electrodes 20, 8 and 23 respectively. Each of these connection terminals 311, 320, 310, and 318 are coupled to the high voltage output circuit 234 to facilitate the delivery of high energy shocking pulses to the heart using one or more of the coil electrodes 8, 20, and 23 and optionally the housing 11. Connection terminals 311, 320, 310 and 318 are further connected to switch matrix 208 such that the housing 11 and respective coil electrodes 20, 8, and 23 may be selected in desired configurations for various sensing and stimulation functions of IMD 10.

The connection terminals 317 and 321 provide electrical connection to the tip electrode 17 and the ring electrode 21 positioned in the right atrium. The connection terminals 317 and 321 are further coupled to an atrial sense amplifier 204 for sensing atrial signals such as P-waves. The connection terminals 326 and 324 provide electrical connection to the tip electrode 26 and the ring electrode 24 positioned in the right ventricle. The connection terminals 307 and 309 provide electrical connection to tip electrode 9 and ring electrode 7 positioned in the coronary sinus. The connection terminals 326 and 324 are further coupled to a right ventricular (RV) sense amplifier 200, and connection terminals 307 and 309 are further coupled to a left ventricular (LV) sense amplifier 201 for sensing right and left ventricular signals, respectively.

The atrial sense amplifier 204 and the RV and LV sense amplifiers 200 and 201 preferably take the form of automatic gain controlled amplifiers with adjustable sensing thresholds. The general operation of RV and LV sense amplifiers 200 and 201 and atrial sense amplifier 204 may correspond to that disclosed in U.S. Pat. No. 5,117,824, by Keimel, et al., incorporated herein by reference in its entirety. Generally, whenever a signal received by atrial sense amplifier 204 exceeds an atrial sensing threshold, a signal is generated on output signal line 206. P-waves are typically sensed based on a P-wave sensing threshold for use in detecting an atrial rate. Whenever a signal received by RV sense amplifier 200 or LV sense amplifier 201 that exceeds an RV or LV sensing threshold, respectively, a signal is generated on the corresponding output signal line 202 or 203. R-waves are typically sensed based on an R-wave sensing threshold for use in detecting a ventricular rate.

In one embodiment of the present invention, ventricular sense amplifiers 200 and 201 may include separate, dedicated sense amplifiers for sensing R-waves and T-waves, each using adjustable sensing thresholds, for the detection of myocardial activation and recovery times. Myocardial activation times may be measured when a signal exceeding an activation time sensing threshold is received by an R-wave sense amplifier included in RV or LV sense amplifiers 200 or 201, causing a corresponding activation time sense signal to be generated on signal line 202 or 203, respectively. Likewise, recovery times may be measured when a signal exceeding a recovery time sensing threshold is received by a T-wave sense amplifier included in RV or LV sense amplifiers 200 or 201, causing a corresponding recovery time sense signal to be generated on signal line 202 or 203, respectively.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion, defibrillation and ESS functions of the IMD 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methodologies known in the art. In one embodiment of the present invention, any available electrodes may be selected by switch matrix 208 for use in determining activation and recovery times employing digital signal analysis methods applied to the EGM signal(s) received from the selected sensing vectors.

The telemetry circuit 330 receives downlink telemetry from and sends uplink telemetry to an external programmer, as is conventional in implantable anti-arrhythmia devices, by means of an antenna 332. Data to be uplinked to the programmer and control signals for the telemetry circuit are provided by microprocessor 224 via address/data bus 218. Received telemetry is provided to microprocessor 224 via multiplexer 220. Numerous types of telemetry systems known for use in implantable devices may be used.

The remainder of the circuitry illustrated in FIG. 2A is an exemplary embodiment of circuitry dedicated to providing ESS, bradycardia pacing, cardioversion and defibrillation therapies. The timing and control circuitry 212 includes programmable digital counters which control the basic time intervals associated with various single, dual or multi-chamber pacing modes or anti-tachycardia pacing therapies delivered in the atria or ventricles. Timing and control circuitry 212 also determines the amplitude of the cardiac stimulation pulses under the control of microprocessor 224.

During pacing, escape interval counters within timing and control circuitry 212 are reset upon sensing of RV R-waves, LV R-waves or atrial P-waves as indicated by signals on lines 202, 203 and 206, respectively. In accordance with the selected mode of pacing, pacing pulses are generated by atrial output circuit 214, right ventricular output circuit 216, and left ventricular circuit 215. The escape interval counters are reset upon generation of pacing pulses, and thereby control the basic timing of cardiac pacing functions, which may include bradycardia pacing, cardiac resynchronization therapy, and anti-tachycardia pacing.

In accordance with the present invention, timing and control circuitry 212 further controls the delivery of extra systolic stimulation pulses at selected extra systolic intervals (ESIs) following either a sensed intrinsic systole or a pacing evoked systole. The ESIs used in controlling the delivery of extra systolic stimuli by IMD 10 are preferably automatically adjusted by IMD 10 based on detected recovery times as will be described in greater detail below. The output circuits 214, 215 and 216 are coupled to the desired electrodes for delivering cardiac pacing or extra systolic stimulation pulses via switch matrix 208.

The durations of the escape intervals used for controlling the timing of stimulation pulse delivery are determined by microprocessor 224 via data/address bus 218. The value of the count present in the escape interval counters when reset by sensed R-waves or P-waves can be used to measure R-R intervals and P-P intervals for detecting the occurrence of a variety of arrhythmias. The microprocessor 224 includes associated ROM in which stored programs controlling the operation of the microprocessor 224 reside. A portion of the memory 226 may be configured as a number of recirculating buffers capable of holding a series of measured intervals for analysis by the microprocessor 224 for predicting or diagnosing an arrhythmia.

In response to the detection of tachycardia, anti-tachycardia pacing therapy can be delivered by loading a regimen from microcontroller 224 into the timing and control circuitry 212 according to the type of tachycardia detected. In the event that higher voltage cardioversion or defibrillation pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the timing and control circuitry 212 by an output circuit 234 via a control bus 238. The output circuit 234 determines the electrodes used for delivering the cardioversion or defibrillation pulse and the pulse wave shape.

In one embodiment, the implantable system may additionally include a physiological sensor for monitoring hemodynamic or myocardial contractile function. The physiological sensor may reside within or on the heart, or endo- or extra-arterially, for sensing a signal proportional to the hemodynamic function of the heart or myocardial contraction or heart wall motion. As such, IMD 10 is additionally equipped with sensor signal processing circuitry 331 coupled to a terminal 333 for receiving an analog sensor signal. A physiological sensor included in the implanted system may be, but is not limited to, a sensor of flow, pressure, heart sounds, wall motion or cardiac chamber volumes. Sensor signal data is transferred to microprocessor 224 via data/address bus 218 such that an index of cardiac hemodynamic or contractile performance may be determined according to algorithms stored in RAM 226. Sensors and methods for determining a cardiac performance index as implemented in the previously-cited '098 patent to Bennett may also be used in conjunction with the present invention.

FIG. 2B is a functional schematic diagram of an alternative embodiment of the IMD 10, which includes dedicated circuitry for detecting myocardial recovery time. Recovery time detection circuitry 100 is provided for receiving one or more EGM or subcutaneous ECG signals via switch matrix 208 and multiplexer 220 on address/data bus 218. In the embodiment of FIG. 2B and with regard to the electrode arrangement of FIG. 1B, connection terminals 328 and 329 are provided for connection to subcutaneous electrodes 30 and 32 incorporated in housing 11, for use in sensing ECG signals. EGM/ECG sensing vectors may be configured from any of the available electrodes via switch matrix 208. Recovery time detection circuitry 100 processes the one or more selected EGM/ECG signals for estimating a recovery time and provides the estimated recovery time to microprocessor 224 for use in controlling device functions that may operate relative to a measured recovery time, such as ESS, anti-tachycardia pacing, or arrhythmia inductions performed during electrophysiological studies. Detected recovery times or major changes in recovery time may be stored for cardiac monitoring purposes.

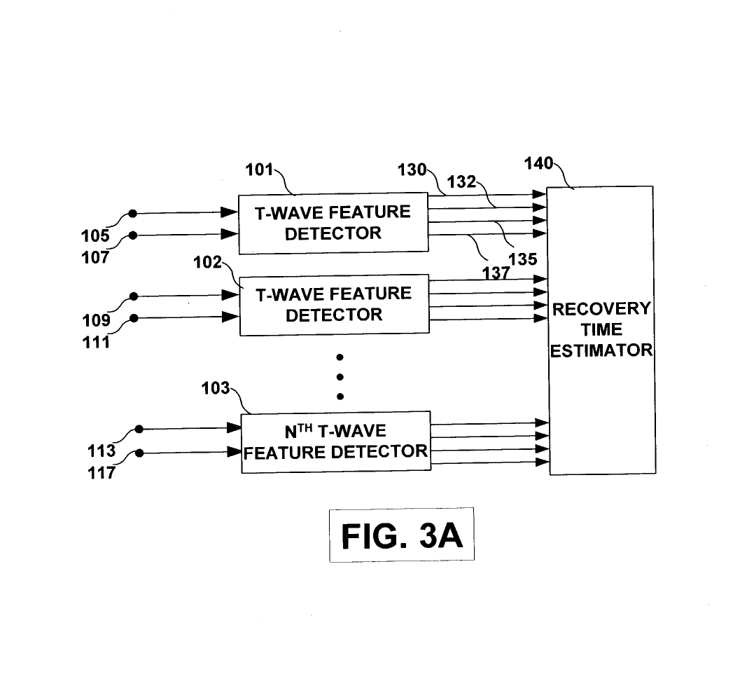

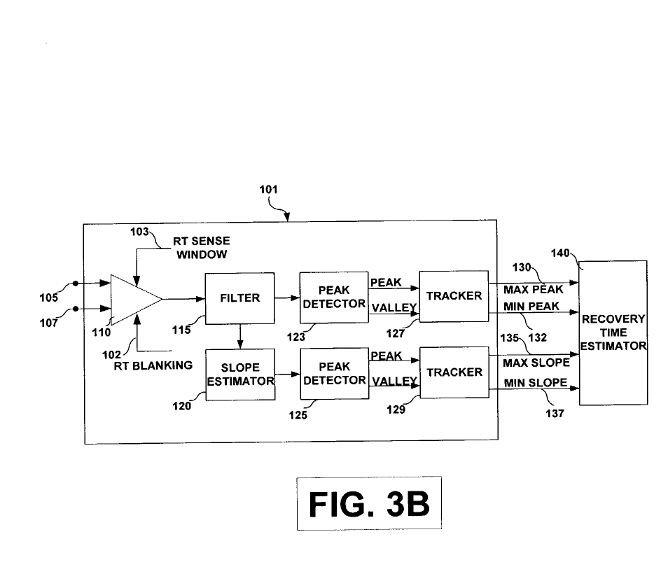

FIG. 3A is a high-level block diagram of the recovery time detection circuitry 100 included in IMD 10 of FIG. 2B. Recovery time detection circuitry 100 includes one or more T-wave feature detectors 101, 102, and 103 and a recovery time estimator 140. T-wave feature detectors 101, 102, and 103 receive EGM or subcutaneous ECG signals from corresponding input lines 105 and 107,109 and 111, and 113 and 117, respectively. Inputs 105 and 107, 109 and 111, and 113 and 117 may correspond to any available electrode pair included in an associated implantable system, including intracardiac unipolar, bipolar, integrated bipolar, or subcutaneous ECG electrode pairs. T-wave feature detectors 101, 102, and 103 process the received EGM/ECG signals to identify fiducial points on the T-wave. When the fiducial points are detected, output signals from T-wave feature detectors 101, 102 and 103 are generated indicating the times of occurrence of detected fiducial points.

The output from T-wave feature detectors 101, 102 and 103 are received as input by recovery time estimator 140. Recovery time estimator 140 estimates the myocardial recovery time based on the time of detected T-wave fiducial points. Fiducial point times are used to corroborate each other in deriving an estimated recovery time. While recovery time detection circuitry 100 is shown in this embodiment to include three T-wave feature detectors 101, 102 and 103, any number of T-wave feature detectors may be used for detecting fiducial T-wave points from a corresponding EGM/ECG signal.

In one embodiment, the fiducial points detected from each EGM/ECG signal include a maximum peak, minimum peak (or valley), maximum positive derivative, and maximum negative derivative. An output signal is generated corresponding to the time of detection of each fiducial point from each T-wave feature detector 101, 102, and 103, which output is received by recovery time estimator 140. Alternative fiducial points may include threshold crossings, baseline crossings, slope threshold crossings, or other identifiable features of the T-wave morphology. In addition to the time of detection, additional information regarding attributes of the detected fiducial point, such as amplitude or magnitude, may be provided as output to recovery time estimator 140.

FIG. 3B is a functional block diagram summarizing signal processing operations performed by the T-wave feature detector of FIG. 3A. Signal processing operations shown in FIG. 3B may be executed by analog or digital circuitry included in recovery time detection circuitry 100. Alternatively, any or all of the signal processing operations summarized in FIG. 3B may be implemented in software stored in RAM 226 and executed by microprocessor 224. T-wave feature detector 101 includes an input amplifier 110 that receives EGM or subcutaneous ECG signals from input lines 105 and 107. Input amplifier 110 may receive a recovery time blanking signal 102, which disables input amplifier 110 during an interval of time following a detected myocardial activation time, during which recovery is not expected to occur. Input amplifier 110 receives an enable signal 103 from the control circuitry of IMD 10, which enables input amplifier 110 to receive the EGM/ECG signal during a recovery time sensing window (RT SENSE WINDOW). Preferably, only a portion of the EGM/ECG signal that includes the T-wave is processed by T-wave feature detector 101. As such, a recovery time sensing window is applied such that input amplifier 110 receives a segment of the EGM/ECG signal that includes the T-wave. As will be described in greater detail below, the recovery time sensing window may be set approximately centered on the T-wave during a calibration procedure. A recovery time sensing window may be applied following both intrinsic and paced events such that the recovery time may be detected during cardiac pacing and during sinus rhythm.

The output of input amplifier 110 is received by a smoothing filter 115. Filter 115 may be a low pass or band pass filter having characteristics matched to the expected frequency content of the myocardial recovery signal. Output from smoothing filter 115 is received by a slope estimator 120 and a peak detector 123. Peak detector 123 detects peaks and valleys of the smoothed EGM/ECG signal. Multiple peaks and valleys may be detected during a recovery time sensing window. Peak/valley tracker 127 receives output from peak detector 123 corresponding to the magnitudes and times of detected peaks and valleys. The peaks and valleys are tracked by peak/valley tracker 127 such that recognition of characteristic peaks and valleys may be made despite beat-to-beat variation in the amplitudes and times of detected peaks and valleys. Peak/valley tracker 127 tracks the peaks and valleys during the recovery time detection window and generates an output signal on corresponding signal lines 130 and 132 corresponding to the time at which the dominant peak and dominant valley occur. The magnitudes of the associated dominant peak and valley may additionally be provided as output.

Output from filter 115 is additionally received by a slope estimator 120. Slope estimator 120 provides the time derivative of the smoothed signal as input to a second peak detector 125. The second peak detector 125 detects the peaks and valleys of the first time derivative, dV/dt, of the EGM/ECG signal. The magnitude and times of the peaks and valleys (multiple peaks and valleys may be detected during the recovery time sensing window) are provided as input to a second peak/valley tracker 129, which tracks the peaks and valleys of the dV/dt signal and determines the dominant maximum peak (maximum positive derivative or slope of the EGM/ECG signal) and the dominant minimum peak (maximum negative derivative or slope of the EGM/ECG signal). The times and optionally the magnitudes of the dominant maximum and minimum slopes are provided as input to the recovery time estimator 140.

As indicated above, recovery time estimator 140 receives the fiducial point times from one or more T-wave feature detectors and corroborates this information to estimate a recovery time. A designated fiducial point time may be used as the recovery time estimate as long as other fiducial point times contribute to a consistent estimate. If a fiducial point time deviates from the estimated recovery time based on a designated fiducial point and other corroborating fiducial point times, the deviant point may be ignored. However, if discrepancies between detected fiducial point times are larger than a predetermined acceptable amount, a recovery time estimate for the current cardiac cycle may be rejected based on uncertain data. If the designated fiducial point time used for estimating recovery time deviates significantly from the recovery time measured for the previous cardiac cycle or a number of previous cardiac cycles, but other fiducial point times support a recovery time estimate consistent with previous recovery times, a recovery time estimate may be made based on one of these other fiducial points and the known previous correlation to the designated fiducial point time.

If major changes occur in the magnitude or time of any of the fiducial points, the recovery time estimate may be rejected. A major change in the T-wave may indicate a change in clinical status or shifting or dislodging of leads. Thus, a major change detected in the magnitude and/or time occurrence of fiducial points may trigger a warning flag that recalibration of the recovery time detection procedure is needed. Recovery time detection calibration procedures will be described in greater detail below.

Major changes in the T-wave, particularly elevation of the S-T segment of an EGM/ECG signal, may indicate myocardial ischemia. Therefore, such changes may be tracked and stored by IMD 10 such that S-T segment changes and T-wave morphology changes are available for review by a clinician or used by IMD 10 to adjust, deliver, or withhold a therapy. For example, detection of an elevated S-T segment may be detected as an indicator of ischemia and used to disable ESS therapy until ischemia is no longer detected or until the therapy is re-enabled by a physician.

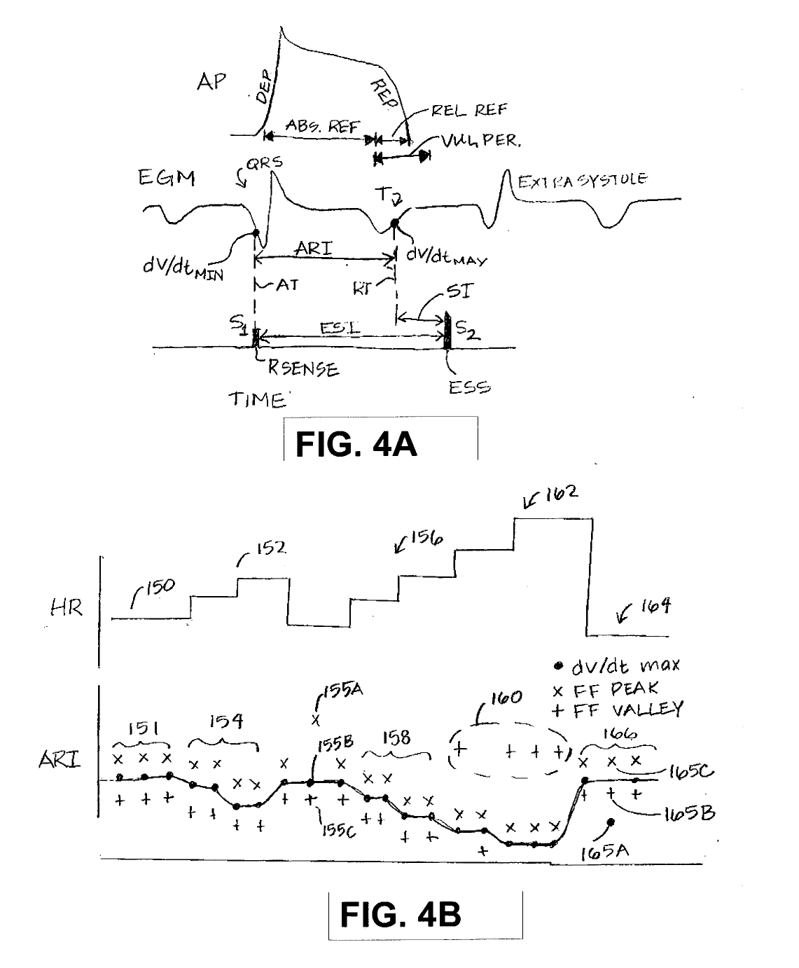

FIG. 4A is an illustration of a representative cardiac action potential signal and a unipolar EGM signal, which may be used for detecting myocardial recovery time. The action potential (AP) is shown in approximate time correspondence to the EGM signal, however, it is recognized that the EGM signal represents the summation of action potentials from a mass of myocardial cells so an exact time correspondence between a single action potential and the EGM is not intended.

The QRS and T-wave portions of the EGM are indicated and approximately correspond to the fast depolarization phase (DEP) and the repolarization phase (REP) of the action potential, respectively. A period of absolute refractory (ABS REF) is associated with the fast depolarization and plateau phases of the action potential. A period of relative refractory (REL REF) is associated with the repolarization phase. The vulnerable period (VUL PER) encompasses the relative refractory period and a period immediately following the relative refractory period.

The activation recovery interval (ARI) is the time interval measured between myocardial activation time (AT) and recovery time (RT) from a selected EGM/ECG signal. In a preferred embodiment, a selected fiducial point for measuring activation time is the time of the maximum negative derivative of the QRS, dV/dtmin. A selected fiducial point for measuring recovery time is the time of the maximum positive derivative, dV/dtmax, of the T-wave. However, it is recognized that other selected points on the QRS and T-wave for measuring activation and recovery time, respectively, may be used in measuring the ARI. For example, in alternative embodiments, activation time may be detected as the time of an activation time sensing threshold crossing, an R-wave peak, an R-wave valley, etc., and a recovery time may be detected as the time of a recovery time sensing threshold crossing, a T-wave peak, T-wave valley, etc.

The timing diagram shown below the EGM signal relates to the control of ESS based on recovery time detection. A sensed R-wave (R SENSE) labeled as a primary systolic event (S1) is shown corresponding to the activation time measured on the QRS of the EGM. The S1 event may be a sinus R-wave or an evoked R-wave following a cardiac pacing pulse. According to one embodiment of the present invention, an extra systolic interval (ESI) is set as the sum of the measured ARI and a safety interval (S1). The ESI is used to time the delivery of extra systolic stimulation pulses. The safety interval added to the ARI is preferably set such that the extra systole evoked by an extra systolic stimulation (ESS) pulse, labeled S2, occurs safely after the vulnerable period following a primary S1 systole but early enough to effectively produce PESP on subsequent cardiac cycles. A safety interval may be equal to or greater than 0, but typically not greater than 100 ms. The safety interval may be a programmable value that remains fixed or may be programmed to vary with other programmable, automatically adjusted, or measured parameters. For example, the safety interval may be set to vary with measured ARIs or with changes in the sensed or paced heart rate.

FIG. 4B is a sample graph of detected activation recovery intervals (ARIs) measured using recovery times detected according to three different fiducial time points at varying heart rates. In this example, the designated fiducial time point for detecting recovery time is based on detecting the maximum positive slope (dV/dt max) of a unipolar EGM. Additional fiducial time points for corroborating the designated fiducial time point in estimating recovery time include, in this example, T-wave peak (FF PEAK) and T-wave valley (FF VALLEY) detected on a far-field EGM signal. At an initial base heart rate 150, stable correlation exists between all three ARIs measured using the three fiducial point times during relatively constant, sinus heart rate. When pacing-induced increases in heart rate are introduced at 152 and 156, the ARIs measured using the three different recovery time fiducial points follow a common trend of decreasing ARI at 154 and 158 with increasing heart rate, as expected.

However, an ARI 155A measured using a recovery time detected based on the far-field peak deviates from the ARIs 155B and 155C measured using the maximum positive derivative and the far field valley. Since the designated fiducial point for detecting recovery time (dV/dt max) produces an ARI 155B consistent with previous ARIs and since the ARI 155C measured using the secondary fiducial point of far-field valley supports the designated fiducial point measurement, the deviant point 155A is ignored.

When the pacing rate is increased further at 162, the ARIs 160 measured based on the recovery time detected using the far-field valley fiducial point become deviant. A change in the T-wave morphology may have occurred. Because the deviation is relatively large, the recovery time estimates are deemed unreliable, and the measured ARIs based on any fiducial points may be disregarded. This change may be stored in IMD memory along with the EGM waveforms for diagnostic purposes.

After the heart rate returns to an intrinsic sinus rate at 164, an ARI 165A based on the designated recovery time fiducial point (dV/dt max) deviates from previous ARI measurements at this heart rate. However, the secondary fiducial points of far-field valley and far-field peak produce consistent ARI measurements 165B and 165C, respectively. The ARI may therefore be estimated based on these secondary ARI measurements 165B and 165C. This estimate may take into account the previously known relation between ARIs measured based on the designated recovery time fiducial point dV/dt max and the secondary recovery time fiducial points, far-field valley and far-field peak.

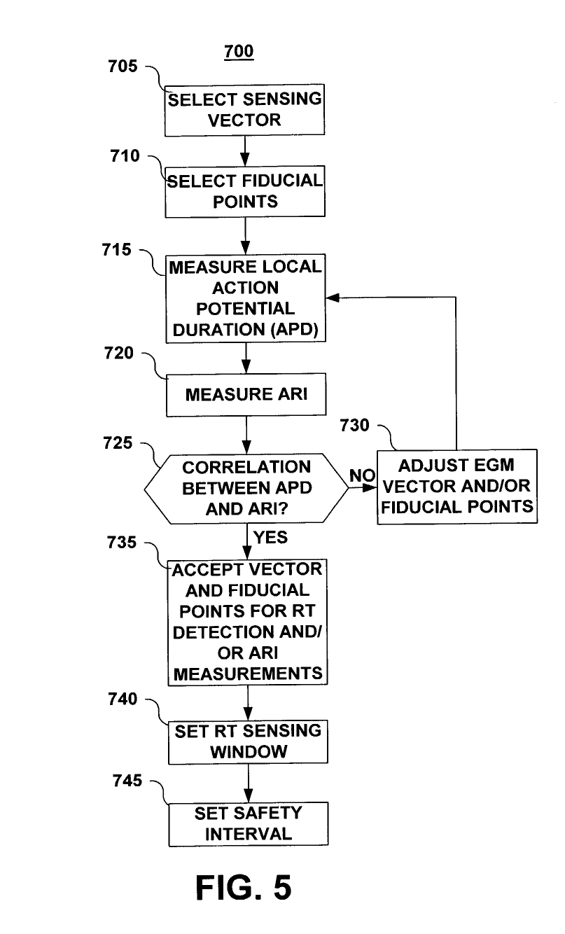

FIG. 5 is a flow chart summarizing steps included in a calibration method for validating an ARI measurement. The calibration method 700 may be performed under clinical supervision for ensuring that the selected sensing vectors and fiducial points used for detecting activation time and recovery time provide an ARI measurement that corresponds to the action potential duration at an ESS site. At step 705, an initial EGM/ECG sensing vector is selected. At step 710, fiducial points are selected for detecting activation time and recovery time on the selected EGM/ECG signal received from the selected sensing vector.

At step 715, a local action potential duration (APD) is measured using a reference electrode system. Any known electrophysiological method for making reliable, acute measurements of local action potential duration may be used. At step 720, an ARI is measured using the selected sensing vector and fiducial points for activation time and recovery time, using the methods described above. Repeated measurements of the local APD and the ARI may be made at steps 715 and 720 at the same or different heart rates to acquire a series of measurements to establish the correlation between the local APD and the ARI measurements. APD and ARI measurements may be performed simultaneously on the same cardiac cycle or sequentially, under stable physiological conditions.

At decision step 725, the local APD(s) measured at step 715 are compared to the ARI(s) measured at step 720 to determine if the ARI measured using the selected sensing vector and fiducial points for activation and recovery time detection is approximately equal to the local APD measurement. If the local APD and ARI measurements differ by more than an acceptable amount, for example by more than a predetermined percentage, the sensing vector and/or the fiducial points selected for measuring activation time and/or recovery time are adjusted at step 730. The local APD measurement(s) are repeated at step 715, and the ARI(s) are measured at step 720 using the adjusted measurement parameters.

Once a satisfactory correlation between the local APD and ARI is obtained, as determined at decision step 725, the currently selected sensing vector and fiducial points for detecting activation time and recovery time are accepted, at step 735, as the operating measurement parameters for measuring the activation time, recovery time and ARI, for use in controlling ESS timing.

At step 740, the measurement of the local APD may be used for setting a recovery time sensing window that is used in searching for the fiducial point on the T-wave. A recovery time sensing window may set such that it is centered approximately on the end point of the local action potential duration. Alternatively, a recovery time sensing window is set to at least begin earlier than the end of the local action potential duration.

The safety interval used in setting the ESI may be set at step 745 based on the measured local APD and any difference in the end point of the APD and the end of the ARI. Thus, the safety interval may be set to account for any deviation between the recovery time measured from a sensed EGM/ECG signal and the time of repolarization measured from the action potential at the ESS site. The safety interval is preferably selected such that an extra systolic stimulation pulse is delivered safely after the vulnerable period. Method 700 may be repeated at varying heart rates such that a unique safety interval may be set for a number of heart rate ranges.

Calibration method 700 may be repeated for each ESS site in multi-site or multi-chamber ESS applications. Steps included in method 700 may be performed only for the purposes of selecting the sensing vector and/or fiducial points for detecting activation time and/or recovery time without setting a recovery time sensing window or safety interval. Alternatively, steps included in method 700 may be performed for setting a recovery time sensing window and/or safety interval, without adjusting sensing vector or fiducial point selections.

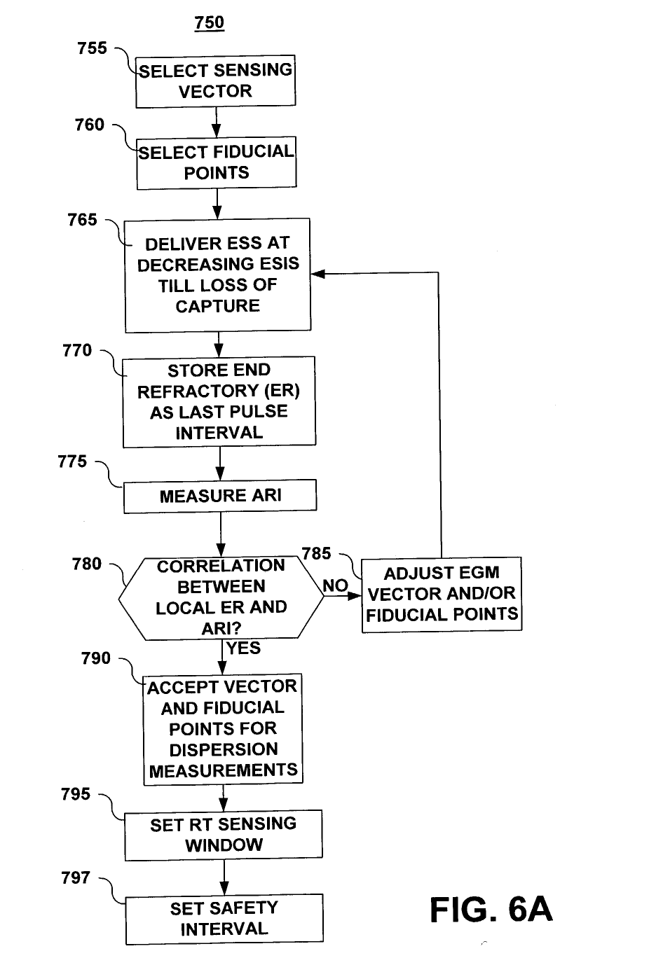

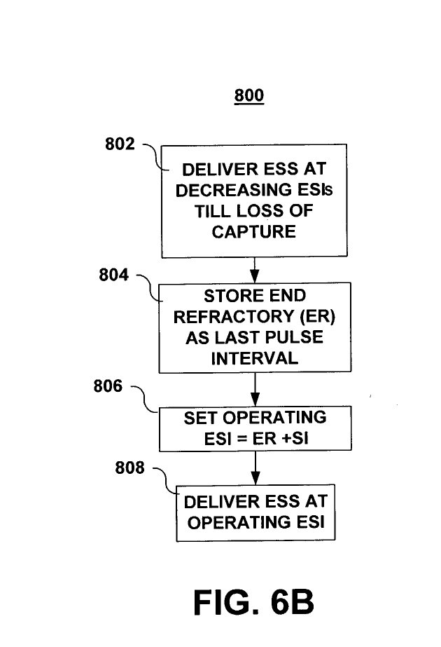

FIG. 6A is an alternative calibration method that may be used for validating an ARI measurement and/or setting a recovery time sensing window and/or safety interval. At steps 755 and 760, respectively, an initial sensing vector and fiducial points for detecting activation time and recovery time for a given ESS site are selected. At step 765, a series of extra systolic pacing pulses are delivered at the ESS site. Extras systolic pacing pulses are inserted in a train of primary pacing pulses at progressively decreasing extra systolic intervals following a primary pacing pulse. Because delivering pacing pulses at short intervals following a previous depolarization can induce an arrhythmia, method 750 is preferably performed under clinical supervision. The interval at which capture is lost is determined as an approximate measure of the end of the local refractory period at the ESS site. At step 770, the last extra systolic pulse interval, which resulted in loss of capture, is stored as the local end refractory time. At step 775, the ARI following an intrinsic or paced primary systole (S1) is measured using the selected sensing vector and fiducial points for activation time and recovery time detection. The local end refractory time is compared to the measured ARI at decision step 780 to verify that the measured ARI is within an acceptable range of the end refractory time.

If the measured ARI and stored end refractory time are not approximately equal, the sensing vector and/or fiducial points for detecting activation time and/or recovery time may be adjusted at step 785. Steps 765 through 785 are repeated until an acceptable correlation between the measured local end refractory time and ARI is obtained, as determined at decision step 780. Alternatively, steps 765 and 770 are performed once and, as long as stable physiological conditions remain, method 750 may loop back to step 775 from step 785 to only repeat ARI measurements at adjusted measurement parameters until satisfactory agreement is reached between an ARI measurement and the previously measured end refractory time. Once a satisfactory correlation between the end refractory time and ARI is obtained, the currently selected sensing vector and fiducial points for detecting activation time and recovery time are accepted at step 790 as the operating measurement parameters for measuring the ARI for a given ESS site, for use in controlling the timing of ESS pulse delivery.

At step 795, a recovery time sensing window may be set based on the measurement of local end refractory time. In one embodiment, the recovery time sensing window is set such that it is centered approximately on the end point of the local end refractory time. Alternatively, a recovery time sensing window is set to at least begin earlier than the end of local refractory. The recovery time sensing window may be used as described previously in detecting recovery time.

The safety interval used in setting the ESI may be set at step 797 based on the measured end refractory time and any difference in the end refractory time and the end of the measured ARI. Thus, the safety interval may be set to account for any deviation between the recovery time measured from a sensed EGM signal and an approximated end refractory time measured at the ESS site. The safety interval is preferably selected such that an extra systolic stimulation pulse is delivered safely after the vulnerable period. Method 750 may be repeated at varying heart rates such that a unique safety interval may be set for a number of heart rate ranges.

Method 750 may be repeated for multiple ESS sites. Steps included in method 750 may be performed only for the purposes of selecting the sensing vector and/or fiducial points for detecting activation time and/or recovery time without setting a recovery time sensing window or safety interval. Alternatively, steps included in method 750 may be performed for setting a recovery time sensing window and/or safety interval without adjusting sensing vector or fiducial point selections.

In alternative embodiments, one or more EGM/ECG signals may be displayed on an external programming device with fiducial points indicated on the signals relative to a measured end refractory time or a measured action potential duration. A clinician may then select the EGM/ECG signal(s) and fiducial point(s) that most closely correspond to the end refractory time or action potential duration for use in measuring ARIs. The clinician may also use this displayed information for setting the recovery time detection window beginning and end and/or use this information for programming a safety interval.

FIG. 6B is a flow chart summarizing steps included in one method for controlling the delivery of ESS based on measuring end refractory time. Method 800 employs an approximate measure of the end of local refractory to set an ESI used during ESS. At step 802, ESS pulses are inserted into a train of primary pacing pulses at progressively decreasing ESIs until loss of capture. At step 804, end refractory is stored as the last pulse interval at which capture was lost. At step 806, the ESI to be applied during ESS therapy is set equal to the end refractory time plus a safety interval. At step 808, ESS is delivered using the ESI set based on an approximate measure of the end of local refractory. Method 800 may be repeated periodically for resetting the ESI as needed, however, method 800 is preferably performed under clinical supervision because of the associated arrhythmia induction risk. Method 800 may be performed at varying heart rates in order to set the ESI for different heart rate ranges. Alternatively, the safety interval used in setting the ESI may be a variable parameter dependent on heart rate, the measured end refractory, or other programmable, automatically adjusted, or measured parameters.

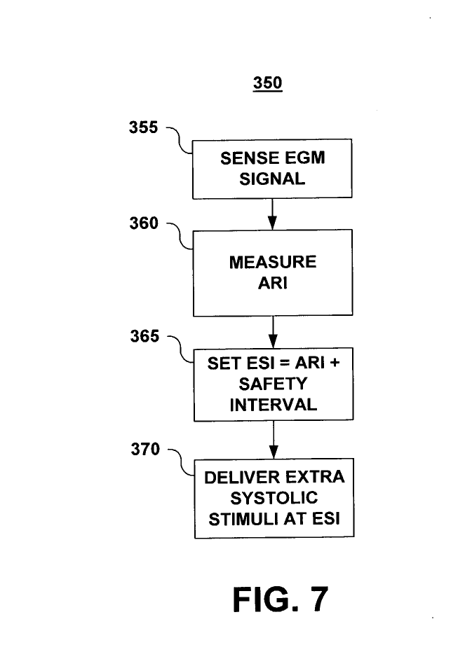

FIG. 7 is a flow diagram providing a general overview of operations included in the present invention for controlling the delivery of extra systolic stimulation relative to a measured recovery time. At step 355, one or more EGM/ECG signals are sensed. In a preferred embodiment, a sensed signal at least includes a unipolar EGM signal sensed at or near the ESS site. As noted previously, the activation-recovery interval (ARI) derived from a unipolar EGM is known to be well correlated to the local action potential duration. Therefore, the recovery time derived from a unipolar EGM signal measured at or very near the site of extra systolic stimulation is expected to provide a reasonably accurate measure of the recovery time. It is contemplated, however, that a reasonably accurate estimate of recovery time that would allow ESS to be safely timed after the vulnerable period could be derived from alternative EGM/ECG sensing vectors including bipolar “tip-to-ring” type sensing vectors, integrated bipolar “coil-to-can” type sensing vectors and even subcutaneous ECG signals, particularly if the correlation to recovery times measured from such alternative sensing vectors to the local recovery time is determined or approximated during a calibration procedure.

At step 360, the ARI is measured from the sensed EGM/ECG signal(s). The activation time is measured as the time at which a fiducial point occurs on the R-wave, which may be an intrinsic R-wave or an evoked R-wave. Recovery time is detected based on the time at which one or more selected fiducial points occur on the T-wave. The ARI is measured as the time interval between the activation time and recovery time measured during a single cardiac cycle. The ARI may optionally be measured for a number of cardiac cycles to determine an average ARI.

At step 365, the operating extra systolic interval is set equal to the measured or averaged ARI plus a predetermined safety interval to ensure that extra systolic stimuli are delivered safely after the vulnerable period. At step 370, ESS pulses are delivered at the operating ESI according to other programmable or predefined ESS control parameters which may include the extra systolic pulse width, pulse amplitude, the ratio of extra systolic stimuli to the normal sinus or paced heart rate, ESS “on” and “off” periods, etc. In one embodiment, the ARI is measured at step 360 on a beat-by-beat or less frequent basis for setting an ESI on a subsequent cardiac cycle(s).

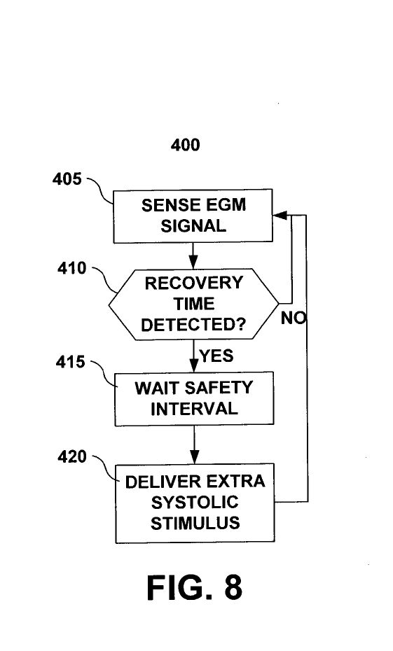

FIG. 8 is a flow chart summarizing an alternative method for dynamically controlling the delivery of extra systolic stimuli based on the detection of recovery time on a beat-by-beat basis. At step 405, an EGM/ECG signal is sensed for detecting recovery time, preferably correlated to the recovery time at the extra systolic stimulation site. If the recovery time can not be detected due to noise or changes in physiologic condition causing unacceptable deviations in fiducial point times, the ESS pulse for that beat may be withheld or delivered at a pre-defined ESI.

Method 400 returns to step 405 to sense the EGM/ECG signal and attempt recovery time detection on the next cardiac cycle. Upon detection of recovery at step 410, a safety interval is initiated at step 415, after which the ESS pulse is delivered at step 420. Method 400 thus allows beat-by-beat detection of recovery time such that the ESS pulses are timed accurately relative to the recovery time detected during each cardiac cycle. It is recognized that an ESS pulse may not be delivered during every cardiac cycle, in which case the recovery time detection and extra systolic stimulation pulse delivery are performed at the desired ratio relative to the intrinsic or paced heart rate (e.g. on every other cardiac cycle, every third cardiac cycle, etc.).

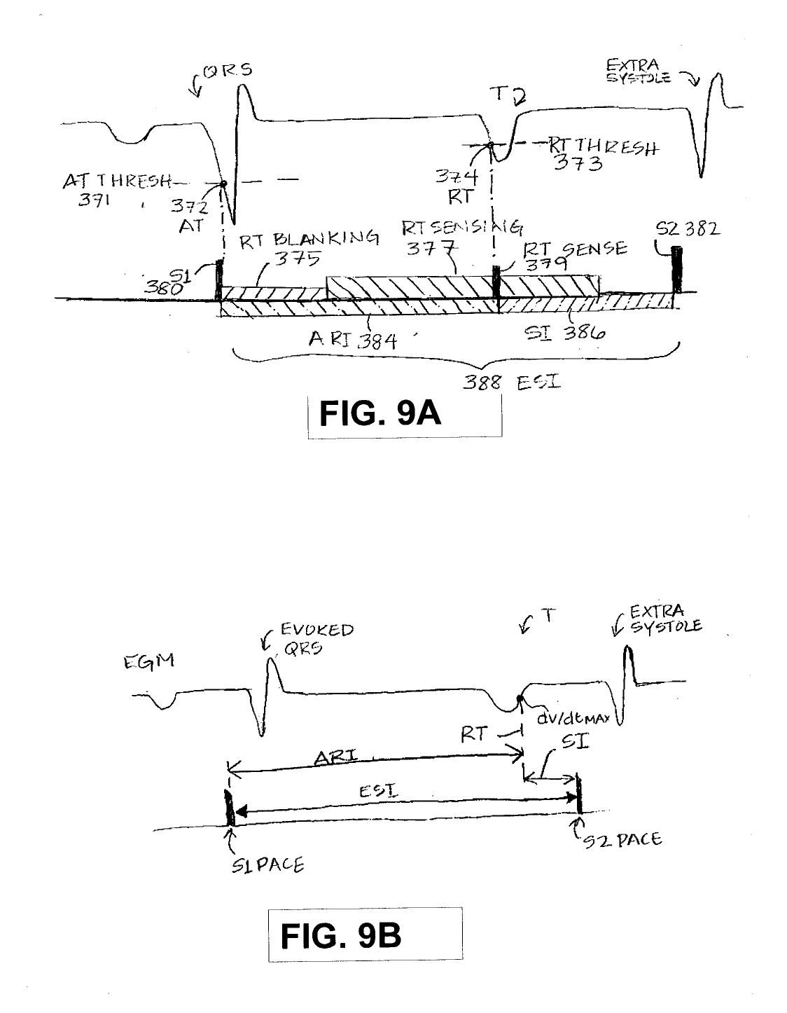

FIG. 9A is a timing diagram shown in temporal relation to an EGM signal to illustrate a methods for detecting activation and recovery times and controlling ESS. As indicated above, a fiducial point on the sensed R-wave on the EGM signal may be detected as the activation time (AT) 372. An S1 event 380, indicated on the timing diagram, is detected corresponding to the detected activation time 372. The activation time 372 may be detected based on any predetermined fiducial point on the R-wave as explained above. In the example shown in FIG. 9A, activation time is detected upon an activation time threshold crossing (AT THRESH, 371).

The recovery time detection method employs the use of timing intervals set relative to the detected activation time for narrowing the search for recovery time. Immediately following a detected activation time, a recovery time blanking period (RT BLANKING, 375) may be applied to recovery time detection circuitry for a time interval following the activation time detection during which recovery is not expected to occur because it is too early after activation. The recovery time blanking period 375 would correspond approximately to the early part of the plateau phase of an action potential.

After the recovery time blanking period 375 has expired, recovery time sensing is enabled during a recovery time sensing window (RT SENSING, 377). In one embodiment, a recovery time sensing window 377 may be positioned in time during a calibration procedure such that it is approximately centered over the expected time occurrence of the T-wave. In an alternative embodiment, the recovery time sensing window 377 may remain enabled from the end of a recovery time blanking period 375 until recovery time (RT, 374) is detected, according to one or more fiducial points on the T-wave or until another activation time is detected. In the embodiment shown in FIG. 9A, a designated fiducial point for detecting recovery time is the time that the T-wave crosses a recovery time threshold 374. If the recovery time is to be detected using digital signal analysis of the sensed EGM signal, the recovery time sensing window may define the beginning and end of an EGM signal segment to be digitized for searching for the fiducial point on the T-wave identified as recovery time.

Recovery time detection is indicated on the timing diagram by a recovery time sense marker (RT SENSE 379). Other fiducial points as described above may be searched for during a recovery time sensing window for use in estimating a recovery time. If a second activation time is detected prior to detecting a recovery time, the recovery time blanking period 375 and recovery time sensing window 377 are reset to begin looking for the next recovery time. The previous intervening recovery time has gone undetected.

If ESS therapy is enabled, an ESS pulse (S2, 382), is delivered upon expiration of a safety interval (SI, 386), which is started upon recovery time detection 379. Alternatively, the ARI 384 measured between the detected activation time 372 and recovery time 374 may be added to a predetermined safety interval 386 for setting an ESI 388 to be used to control the timing of an S2 pulse on the next cardiac cycle.

The ESI applied during ESS following an intrinsic R-wave, also referred to as “triggered” or “coupled” pacing, and the ESI applied during ESS following an-evoked R-wave, also referred to as “paired pacing”, may be uniquely defined to account for differences in intrinsic and paced ARIs. FIG. 9B is an illustration of a representative EGM signal and a timing diagram illustrating the operation of ESS during paired pacing. In this embodiment, the ARI during cardiac pacing is measured as the time interval between a primary pacing pulse (S1 PACE) and a consecutively measured recovery time (RT). Alternatively, the ARI may be measured as the time interval between a fiducial point on the evoked QRS signal and a consecutively measured recovery time. The ESI during paired pacing is set as the sum of the measured ARI and a safety interval (SI). In operation, the ESI for paired pacing is applied following an S1 pacing pulse and is immediately followed by an S2 pacing pulse (S2 PACE). If intrinsic cardiac activity interrupts cardiac pacing during demand pacing modes, the ESI for coupled pacing, set according to the methods described above in conjunction with FIG. 9A, is applied. Thus, the ESI applied following a paced S1 event and the ESI used following an intrinsic S1 event may be the same or uniquely defined.

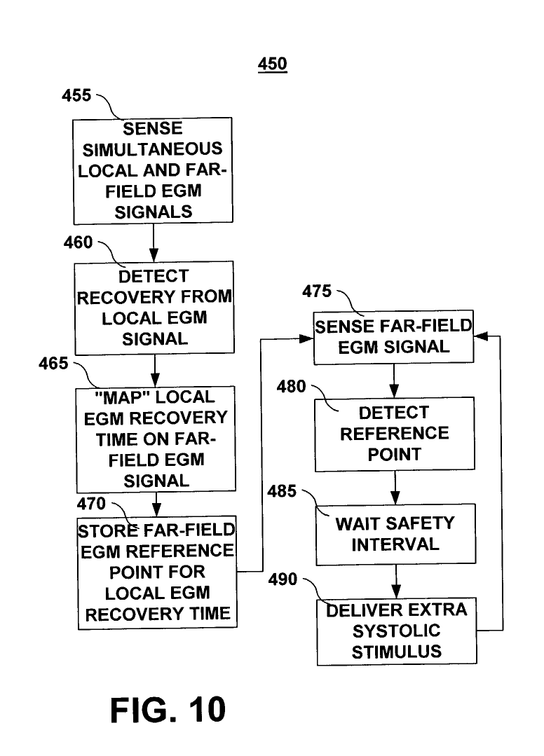

FIG. 10 is a flow diagram of yet another embodiment for controlling the delivery of ESS based on detection of recovery time. Depending on the lead system available, it may be desirable to sense a far-field EGM or subcutaneous ECG signal during ESS operations rather than sensing a near-field EGM signal. For example, during paired pacing, the local EGM signal may be contaminated by post-pace polarization artifact. However, depending on the far-field vector configurations available, the recovery time at the ESS site may not be readily ascertained from a far-field signal for use in dynamically controlling the ESI on a beat-by-beat basis. Method 450 of FIG. 10 provides a “mapping” procedure in which the local recovery time detected from a local EGM signal is mapped in time on a far-field EGM/ECG signal providing a reference point on the far-field signal (or ECG signal) corresponding to local recovery time at the ESS site.

Method 450 begins at step 455 by selecting and simultaneously sensing a local EGM signal and a far-field EGM/ECG signal. At step 460, local recovery is detected on the local EGM signal according to the methods described previously. At step 465, the time point associated with local recovery on the local EGM signal is identified on the far-field signal so as to “map” the local recovery time on the far-field signal. This time point is stored at step 470 as the far-field reference point for local recovery at the ESS site. The time point may be stored relative to another fiducial point on the T-wave of the far-field signal, such as a maximum or minimum peak, maximum or minimum derivative, etc. The reference time point may alternatively be stored relative to R-wave detection on the far-field signal. If the mapping procedure is performed during cardiac pacing, the reference time point may be stored relative to the delivery of the pacing pulse.

Having stored a reference point representing local recovery on the far-field EGM/ECG signal, far-field EGM/ECG sensing is performed during ESS operations at step 475 for timing the delivery of ESS pulses. At step 480, the reference point is identified on the sensed far-field EGM/ECG. A predefined safety interval is allowed to time out at step 485, which ensures that the extra systolic stimulus occurs safely after the vulnerable period. At step 490, the extra-systolic stimulus is delivered at an ESI that is adjusted beat-by-beat according to the detected reference point on the far-field EGM/ECG. Steps 475 through 490 are continuously repeated as long as ESS delivery is enabled. Steps 455 through 470 for mapping a local recovery time on the far-field EGM/ECG signal may be repeated periodically in order to maintain an accurate reference point representing the local recovery time at an ESS site on the far-field EGM/ECG signal.

It is contemplated that methods for automatically adjusting the ESI based on recovery time detection may further include adjustments made to optimize hemodynamic or mechanical heart function on post-extra systolic heartbeats. A minimum allowable ESI may be set based on the measured recovery time. The ESI may be adjusted to intervals longer than the minimum limit in order to maximize hemodynamic or mechanical heart function parameters measured during post-extra systolic beats using a physiological sensor.

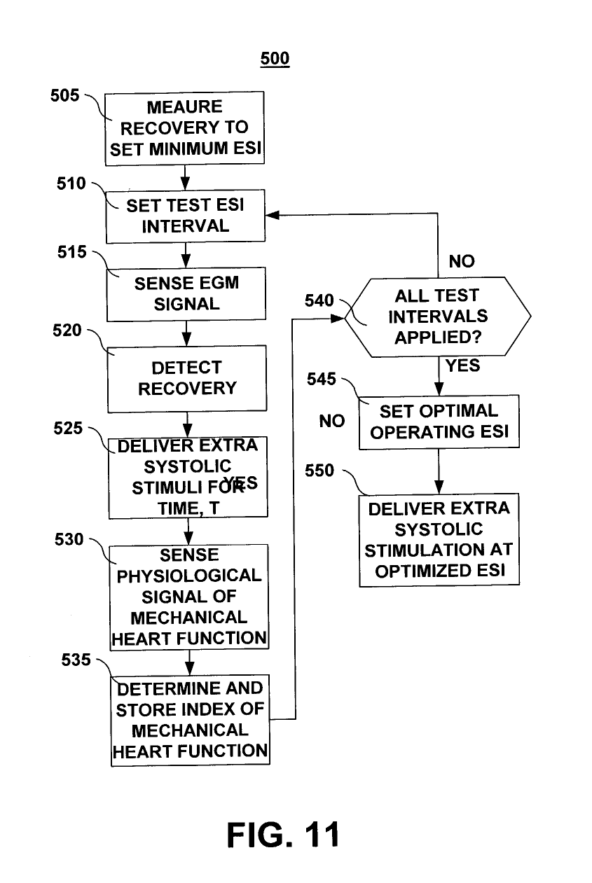

FIG. 11 is a flow chart summarizing steps included in a method for automatically adjusting an ESI based on both recovery time and one or more indices of mechanical heart function, which may be hemodynamic indices or indices of myocardial contraction performance. At step 505, the recovery time is measured according to the methods described previously to set a minimum ESI. The minimum ESI is set as an interval extending just beyond the recovery time such that ESS pulses are safely delivered after the vulnerable period.

At step 510, an iterative procedure for identifying an optimal ESI begins by setting the ESI to the first of a set of test ESI values. The test ESI values may be a predetermined number of values at increasing increments from and including the minimum ESI determined at step 505. The test ESIs may be applied in a random order or in a generally increasing or decreasing order. After setting the first test interval at step 510, a selected EGM/ECG signal for controlling ESS timing is received at step 515, and ESS is delivered at step 525 at the test ESI for a period of time, T, or a predetermined number of cardiac cycles. A predetermined test time interval T may be on the order of a few seconds to few minutes and is preferably long enough to allow hemodynamic stabilization to be reached after applying the new ESI.

After the stabilization period, ESS continues at the applied test ESI and one or more physiological signals relating to hemodynamic function or mechanical contractile function are sensed at step 530. Examples of physiological sensors that may be used for assessing hemodynamic or contractile heart function include, but are not limited to an atrial, ventricular or arterial blood pressure sensor, an intra-cardiac or arterial blood flow sensor, electrodes for measuring thoracic or cardiac impedance for estimating cardiac output, a heart sound sensor, or an accelerometer or other sensor of heart wall motion. Hemodynamic or myocardial contractile function may be measured during one or more post-extra systolic beats or may be averaged over a predetermined period of time or number of cardiac cycles during applied ESS.

At step 535, an index of hemodynamic or contractile function is determined from the sensed signal(s) and stored with the corresponding test ESI. In one embodiment, a cardiac performance index may be determined as generally described in the previously incorporated '098 patent to Bennett et al. At step 540, method 500 determines if all test ESIs have been applied. If not, method 500 returns to step 510 to apply the next test interval and obtain and store an index of hemodynamic or mechanical heart function associated with that test interval. Once all test intervals have been applied, the operating ESI is set at step 545 to an optimal setting that is equal to or greater than the minimum ESI allowed based on recovery time detection and which results in maximal hemodynamic or myocardial contractile function.

At step 550, ESS is delivered at the optimized ESI according to the programmed ESS operating parameters. It is recognized that an optimal ESI following sensed intrinsic R-waves and an optimal ESI following pacing-evoked R-waves may be individually determined according to method 500 and applied during coupled and paired ESS, respectively.

It is further recognized that methods presented herein for applying ESS at a safe interval following recovery measured at one stimulation site may be expanded to delivering ESS at two or more stimulation sites based on measuring recovery at each of the stimulation sites. One effect of ESS is that depolarization wavefront propagation can be slowed when extra systolic stimuli are applied. During multi-site ESS, propagation of the activation wavefront from an ESS site to another pacing site or ESS site may be slowed. Changes in activation propagation may alter the dispersion of the time of electrical activation and recovery over the heart during a given cardiac cycle. Increased dispersion of electrical activation, recovery and the interval between activation and recovery creates an important substrate for arrhythmias. Repolarization dispersion as well as the orientation of repolarization gradients may be important determinants of the vulnerability to re-entrant tachycardias. A system and method for monitoring electrical dispersion is generally disclosed in co-pending U.S. Pat. Appl. No. P11215 to Burnes, incorporated herein by reference in its entirety. During multi-site ESS, it is desirable to maintain or reduce recovery dispersion.

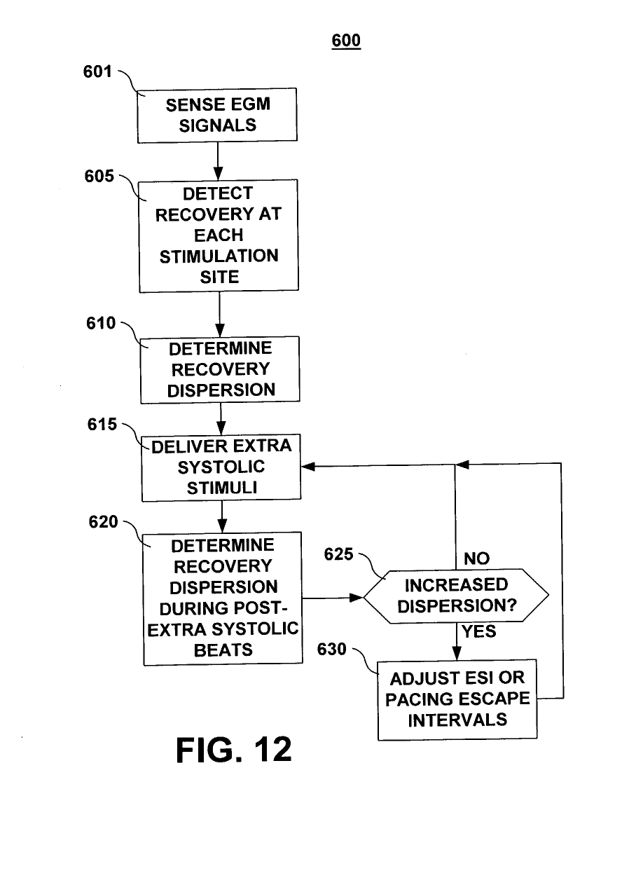

FIG. 12 is a flow chart summarizing a method for applying multi-site ESS in which relative recovery times between stimulation sites are maintained or recovery time dispersion is reduced. At step 601, selected EGM signals are received from which recovery times at each of a number of ESS sites are measured at step 605. The recovery time dispersion is determined at step 610 by determining the differences between recovery times at each of the stimulation sites during a given cardiac cycle or sets of sequentially recorded similar beats. A detailed method for measuring recovery time dispersion using an implanted system is provided in co-pending U.S. Pat. Appl. P11215 to Burnes.

At step 615, multi-site ESS is delivered at two or more selected sites at ESIs determined according to methods described previously for ensuring the extra systolic stimuli are delivered safely after recovery at each ESS site. At step 620, recovery dispersion is measured during post-extra systolic beats. At decision step 625, a determination is made whether the post-extra systolic dispersion is increased compared to the pre-ESS dispersion measurement made at step 610 or is greater than a predetermined acceptable level. If recovery dispersion is unchanged or below an acceptable level, method 600 returns to step 615 and ESS continues at the current ESIs.

However, if the dispersion is high or increased compared to the pre-ESS dispersion measure, one or more ESIs and/or a cardiac pacing interval, such as a V-V interval, may be adjusted at step 630 in order to reduce the dispersion of recovery. ESS is delivered at the adjusted intervals at step 615, and the dispersion of recovery is re-determined at step 620. Adjustments to the ESI and/or a cardiac pacing interval can be made at step 630 until the dispersion of recovery is reduced to an acceptable or pre-ESS dispersion level. Measurement of recovery dispersion may be performed periodically in order to ensure that multi-site ESS has not unfavorably altered recovery dispersion.

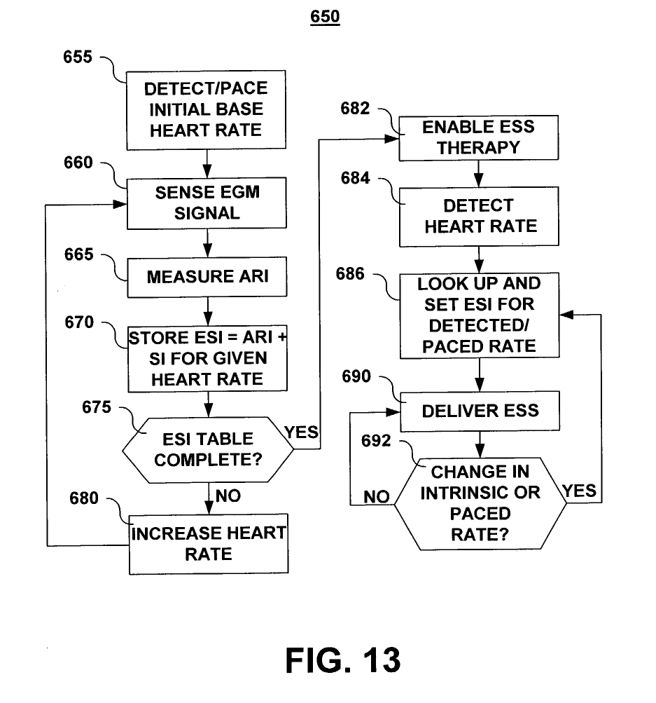

FIG. 13 is a flow chart summarizing a method for controlling ESS according to previously-determined ESIs based on ARIs measured over a range of heart rates. In this embodiment, ARIs are measured at different heart rates such that an ESI may be determined and stored for each heart rate, allowing automatic ESI adjustment with variations in the intrinsic or paced heart rate without having to re-measure the ARI and calculated a new ESI. Method 650 shown in FIG. 13 includes steps for compiling a “look-up” table of ESIs and steps for delivering ESS at ESIs stored in the “look-up” table.

At step 655, the initial heart rate is determined. The heart rate may be an intrinsic, sinus rate or a paced rate. At step 660, an EGM/ECG signal selected for detecting recovery is sensed, and the ARI is measured at step 665 according to methods described previously. At step 670, an ESI is stored for the given heart rate, or a pre-defined range that includes the detected/paced heart rate, as the sum of the measured ARI and a safety interval, SI. The safety interval may be a programmable setting or may be a function of or vary with another programmed or measured parameter, such as the measured ARI or cardiac cycle length.

At step 675, method 650 determines if an ESI look-up table is complete for a number of heart rates or heart rate ranges. If the table is not complete, the heart rate is increased at step 680. The steps performed for generating an ESI look-up table may be performed under clinical supervision such that heart rate increases at step 680 are exercise-induced, for example, by controlled treadmill or stationary bicycle exercise. A number of heart rate zones may be tested by asking the patient to exercise until the heart rate has reached a certain level, then asking him/her to maintain the same level of exertion while steps 660 through 675 are repeated for determining an appropriate ESI for the given heart rate zone. Alternatively, heart rate increases may be controlled by increasing the pacing rate in stepwise increments. Pacing induced increases in heart rate for generating an ESI look-up table may be performed automatically, with or without clinical supervision. Two or more heart rated or heart rate zones may be included in the ESI look-up table.

Once the look-up table is complete, ESS therapy is enabled at step 682. At step 684, the current heart rate, intrinsic or paced, is detected or identified, and the ESI is set at step 686 based on the value stored in the look-up table for the corresponding heart rate. ESS is delivered at step 690 at the ESI previously determined safe and appropriate for the given heart rate or heart rate zone. Throughout ESS delivery, the intrinsic/paced heart rate is monitored for shifts to different heart rate zones as indicated at decision step 692. If the intrinsic or paced rate increases or decreases to a different heart rate zone, method 650 returns to step 686 to adjust the ESI to the stored look-up table ESI value corresponding to the new heart rate zone. ESS is applied at the adjusted ESI at step 690.

Steps 655 through 680 for compiling an ESI look up table may be performed on a period basis in order to update the stored ESIs according to changes in ARIs at varying heart rates that may occur with changes in disease state, medical therapy or other physiologic conditions.

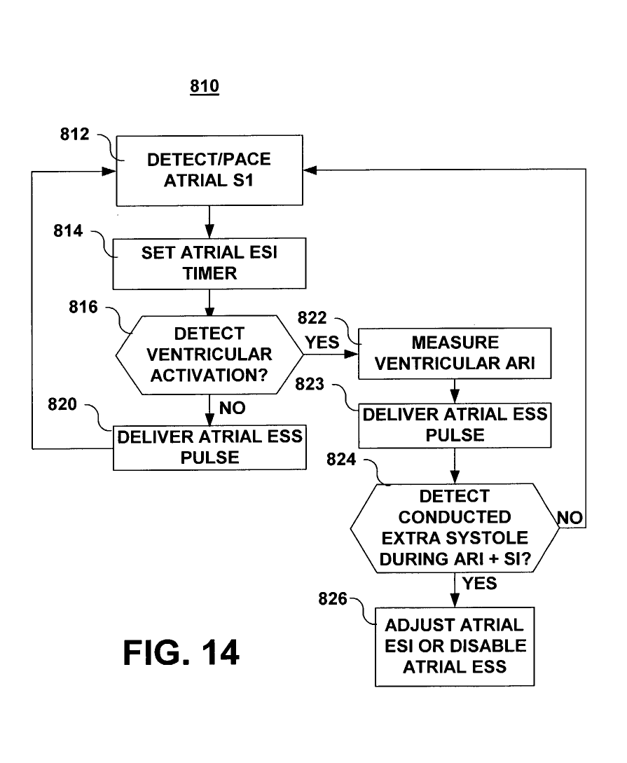

FIG. 14 is a flow chart summarizing steps included in a method for controlling the delivery of atrial ESS. It is recognized that methods described above for automatically adjusting a ventricular ESI based on detecting recovery time in the ventricle may be modified for automatically adjusting an atrial ESI based on detecting recovery in an atrial chamber and/or measuring an atrial ARI. An ESS pulse delivered in the atrium may be conducted to the ventricles to produce the mechanical augmentation due to PESP in both the atria and ventricles. However, in order to avoid inducing a ventricular arrhythmia due to rapid conduction of an atrial extra systolic stimulus to the ventricle during the ventricular vulnerable period following a primary ventricular systole, it is desirable to monitor the ventricular rhythm and inhibit or adjust the atrial ESS pulse if the atrial systole and extra-systole both conduct to the ventricles, and if the conducted extra-systole occurs during the ventricular ARI plus safety interval.

Method 810 begins at step 812 by detecting an intrinsic atrial systole or delivering a primary atrial pacing pulse, referred to as the atrial S1 event. At step 814, an atrial ESI timer is set corresponding to a previously determined atrial ESI. The atrial ESI may be set based on measurements of an atrial ARI. At step 816, method 810 monitors the ventricular EGM/ECG signals to detect any ventricular activation occurring during the atrial ESI. The atrial S1 event may be conducted to the ventricles resulting in a ventricular S1 activation. If ventricular activation is not detected during an atrial ESI, the scheduled atrial ESS pulse is delivered at step 820, and method 810 returns to step 812 to continue atrial ESS delivery.

If, however, a ventricular S1 activation is detected during the atrial ESI (at decision step 816), method 810 begins monitoring for the ventricular S1 recovery time at step 822. The atrial ESS pulse is delivered as scheduled upon expiration of the atrial ESI at step 823. At step 824, the device monitors the ventricular EGM/ECG signal for a conducted extra systole occurring prior to the ventricular S1 RT or during a safety interval immediately following the ventricular RT. If the atrial extra systole is not conducted to the ventricles during the ventricular S1 ARI plus safety interval, method 810 returns to step 812 to continue delivering atrial ESS.

If, however, the atrial extra systole is conducted to the ventricles such that a second ventricular activation is detected during the ventricular S1 ARI plus safety interval (decision step 824), the atrial ESI may be adjusted at step 826 such that on the next cardiac cycle, the conducted extra systole will occur after the ventricular S1 ARI plus safety interval. By lengthening the atrial ESI, a conducted extra systole from the atrium will depolarize the ventricles after the vulnerable period following a primary ventricular systole. Alternatively, atrial ESS may be disabled at step 826 to avoid any risk of rapidly conducted extra systoles occurring during the ventricular vulnerable period.

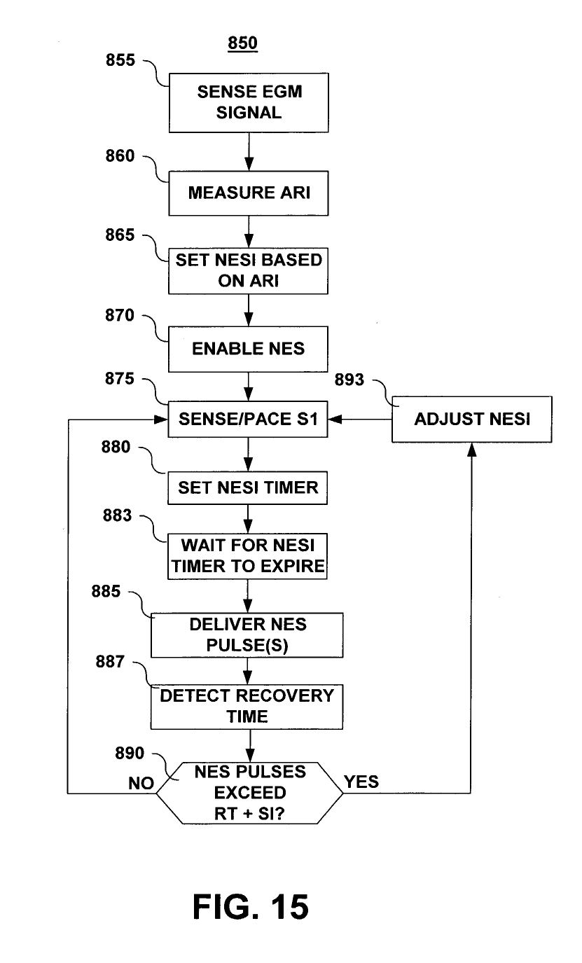

FIG. 15 is a flow chart summarizing steps included in a method for controlling the delivery of non-excitatory stimulation pulses based on detecting recovery time. It is contemplated that methods described herein for detecting myocardial recovery may be usefully practiced in controlling the delivery of other types of electrical stimulation therapy that requires careful timing relative to the myocardial refractory and vulnerable periods. As noted previously, non-excitatory stimulation (NES) delivered during myocardial refractory has been proposed as a method for modulating cardiac contractility. Measurements of ARI using the methods and apparatus provided by the present invention may be used in controlling the timing of NES pulses.

Method 850 begins at step 855 by sensing a selected EGM or ECG signal and measuring the ARI from the EGM or ECG signal at step 860. A non-excitatory stimulation interval (NESI), at which NES pulse delivery is initiated following a paced or intrinsic event, is set at step 865 based on the measured ARI and the number of NES pulses to be delivered. A NESI may be set as a predetermined percentage of the measured ARI or as the measured ARI less a predetermined safety interval used to ensure that NES pulses are delivered prior to the end of myocardial refractory.

At step 870, NES therapy is enabled. A primary systole, S1, is either sensed or paced at step 875. Upon pacing or sensing a primary systole, a NESI timer is set at step 880. Method 850 waits for the NESI timer to expire at step 883 after which the NES pulses are delivered at step 885.

Method 850 searches for the recovery time at step 887. Recovery may occur before, during or after the NES pulses, therefore monitoring for recovery time may be performed before and after NES pulse delivery and may occur during NES pulse delivery depending on the electrodes used for recovery time sensing and NES. At step 890, method 850 determines if the timing of NES pulses exceeded the recovery time plus a safety interval. Alternatively, recovery time may be searched for only after NES pulses are delivered, and, if recovery time is not detected, recovery is assumed to have occurred prior to or during NES pulse delivery.

If the timing of NES pulses is determined to occur within the recovery time plus a safety interval at decision step 890, method 850 returns to step 875 to continue NES therapy. If the timing of NES pulses is determined to have exceeded the recovery time plus a safety interval at decision step 890, the NESI and/or the number of NES pulses is adjusted at step 893. As long as NES remains enabled, method 850 returns to step 875 to sense/pace the next primary systole. Step 893 for adjusting the NESI and/or number of NES pulses according to a detected recovery time may optionally be performed on a beat-by-beat or less frequent basis or only when NES pulses exceed the recovery time plus safety interval as shown in FIG. 15.

Thus, an implantable system and associated methods have been described for measuring recovery time based on sensed EGM/ECG signals and for controlling the timing of cardiac stimulation pulses relative to a detected recovery time. The present invention allows the hemodynamic benefits of post-extra systolic potentiation to be gained in an electrical stimulation therapy for treating cardiac mechanical insufficiency while minimizing the potential risk of inducing arrhythmias due to extra systolic stimulation pulses delivered during the vulnerable period. While the present invention has been described according to specific embodiments presented herein, these embodiments are intended to be exemplary, not limiting, with regard to the following claims.

1. A method for providing cardiac electrical stimulation therapy, comprising:
sensing a signal associated with electrical activity of a heart;
detecting a myocardial recovery condition in the signal; and
delivering an extra systolic stimulation pulse at a predetermined time interval after detecting the recovery condition.

2. A method according to claim 1, wherein said signal is based on an electrogram.

3. A method according to claim 2, wherein said signal is a signal from an atrial chamber.

4. A method according to claim 2, wherein said signal is a signal from a ventricular chamber.

5. A method according to claim 1, wherein said predetermined time interval is based at least in part on a presently detected heart rate.

6. A method according to claim 1, further comprising:
detecting a heart rate; and
applying a different predetermined amount of time for a first range of heart rate than a second range of heart rate.

7. A method according to claim 1, wherein the delivery of the extra systolic stimulation pulse occurs on a next-occurring cardiac cycle.

8. A method according to claim 1, wherein the predetermined time interval comprises a range having a minimum allowable value.

9. An apparatus for providing cardiac electrical stimulation therapy, comprising:
means for sensing a signal associated with electrical activity of a heart;
means for detecting a myocardial recovery condition in the signal; and
means for delivering an extra systolic stimulation pulse at a predetermined time interval after detecting the recovery condition.

10. An apparatus according to claim 9, wherein said signal is based on an electrogram derived from at least two discrete intracardiac electrodes or a pair of discrete subcutaneous electrodes.

11. An apparatus according to claim 10, wherein said signal is a signal sensed from an atrial chamber by at least a pair of electrodes and at least one of said pair of electrodes comprises:
an indifferent electrode;
a surface electrode coupled to a device housing;
a mono-polar electrode;
a bi-polar electrode;
an intracardial electrode;
an epicardial electrode;
a subcutaneous electrode.

12. An apparatus according to claim 10, wherein said signal is a signal sensed from a ventricular chamber by at least a pair of electrodes and at least one of said pair of electrodes comprises:
an indifferent electrode;
a surface electrode coupled to a device housing;
a mono-polar electrode;
a bi-polar electrode;
an intracardial electrode;
an epicardial electrode;
a subcutaneous electrode.

13. An apparatus according to claim 9, wherein said predetermined time interval is based at least in part on a presently detected heart rate.

14. An apparatus according to claim 9, further comprising:
means for detecting a heart rate; and
means for applying a different predetermined amount of time for a first heart rate than a second heart rate.

15. An apparatus according to claim 9, wherein the delivery of the extra systolic stimulation pulse occurs on a next-occurring cardiac cycle.

16. An apparatus according to claim 9, wherein the predetermined time interval comprises a range having a minimum allowable value.

17. An apparatus according to claim 9, wherein said means for detecting a recovery condition comprises a recovery time detection window and said recovery time detection window further comprises at least one T-wave event.

18. An apparatus according to claim 17, further comprising a threshold component to said recovery time detection window, and wherein a number of crossing events of said threshold component is used in estimating a myocardial recovery time.

19. An apparatus according to claim 17, further comprising a means for recognizing a morphological waveform feature and wherein said means identifies T-wave features and provides an output signal related to identified T-wave features.

20. An apparatus according to claim 19, wherein said means for recognizing comprises digital signal processing circuitry.

21. A computer readable medium for storing computer instructions for performing a method, comprising:
instructions for sensing a signal associated with electrical activity of a heart;
instructions for detecting a myocardial recovery condition in the signal; and
instructions for delivering an extra systolic stimulation pulse at a predetermined time interval after detecting the recovery condition.

22. A medium according to claim 21, wherein said signal is based on an electrogram derived from at least two discrete intracardiac electrodes or a pair of discrete subcutaneous electrodes.

23. A medium according to claim 22, wherein said signal is a signal from an atrial chamber.

24. A medium according to claim 22, wherein said signal is a signal from a ventricular chamber.

25. A medium according to claim 21, wherein said predetermined time interval is based at least in part on a presently detected heart rate.

26. A medium according to claim 21, further comprising:
instructions for detecting a heart rate with at least one electrode coupled to a heart rate detection circuit; and
instructions for applying a different predetermined amount of time for a first heart rate than a second heart rate.

27. A medium according to claim 21, wherein the delivery of the extra systolic stimulation pulse occurs on a next-occurring cardiac cycle.

28. A medium according to claim 21, wherein the predetermined time interval comprises a range having a minimum allowable value.

29. A medium according to claim 21, wherein said means for detecting a recovery condition comprises a recovery time detection window and said recovery time detection window further comprises at least one T-wave event on at least one EGM vector.

30. A medium according to claim 29, further comprising instructions for establishing a threshold component to said recovery time detection window, and wherein a number of crossing events of said threshold component is used in estimating a myocardial recovery time.

31. A medium according to claim 29, further comprising a means for recognizing a morphological waveform feature and wherein said means identifies T-wave features and provides an output signal related to identified T-wave features.

32. A medium according to claim 30, wherein said means for recognizing comprises digital signal processing circuitry

33. A method according to claim 3, wherein if an atrial extra systole occurs during a ventricular recovery interval or a ventricular safety interval, further comprising, either:
lengthening the predetermined time interval; or
ceasing delivery of the therapy.

34. A method for providing multiple-site cardiac electrical stimulation therapy, comprising:
sensing a signal associated with electrical activity of a heart, wherein said signal includes an extra systolic stimulation component;
detecting a recovery condition in the signal based at least in part on the extra systolic stimulation component; and
delivering at least two extra systolic stimulation pulses to at least two different locations of a single cardiac chamber at an individually and a physiologically coordinated predetermined time interval after detecting the recovery condition so that dispersion of recovery is not dispersed among said at least two different locations.

35. A medium according to claim 29, further comprising a means to adjust the predetermined time interval of extra systolic stimulation based on a dispersions in recovery times as measured between at least two EGM vectors.

36. An apparatus according to claim 17, wherein the T-wave event is identified on a selected vector based on where a T-wave event occurs on a different vector.

37. A method for providing cardiac electrical stimulation therapy, comprising:
sensing a first signal associated with electrical activity of a heart;
detecting an electrical myocardial recovery condition in the signal;
sensing a second signal associated with a heart function;
detecting an index of heart function;
delivering an extra systolic stimulation pulse after expiration of a time interval following detecting the electrical myocardial recovery condition; and
producing a maximized index of heart function.

38. A method according to claim 37, wherein said heart function is related to a mechanical aspect of the heart function.

39. A method according to claim 37, wherein said heart function is related to a hemodynamic aspect of the heart function.

40. A method according to claim 37, wherein said heart function is related to a myocardial contractile performance.

41. A method for providing cardiac electrical stimulation therapy, comprising:
sensing a signal associated with electrical activity of a heart;
detecting a myocardial recovery condition in the signal; and
controlling the delivery of non-excitatory stimulation pulses after detecting the recovery condition.

42. A method according to claim 41, wherein the non-excitatory stimulation pulses are terminated upon detection of the myocardial recovery condition.

43. A method according to claim 41, wherein the non-excitatory stimulation pulses for the next cardiac cycle or cardiac cycles are delivered so as to terminate within the time interval of the myocardial recovery condition.

44. A method for providing cardiac electrical stimulation therapy, comprising:
sensing a signal associated with electrical activity of a heart, wherein said signal includes at least a T-wave component;
detecting a recovery condition in the signal based at least in part on the T-wave component; and
delivering an extra systolic stimulation pulse at a predetermined time interval after detecting the recovery condition.
